(19) United States
(12) Patent Application Publication
(10) Pub. No.: US 2002/0035111 A1
(43) Pub. Date: Mar. 21, 2002

(54) Method of inhibiting neoplastic cells with tetracyclic pyrido[3,4-B] indole derivatives

(76) Inventors: Rifat Pamukcu, Spring House, PA (US); Gary Piazza, Doylestown, PA (US)

Correspondence Address: Cell Pathways, Inc., 702 Electronic Drive, Horsham, PA, 19044, US

(21) Appl. No.: 09/974,271

(22) Filed: Oct. 10, 2001

Related U.S. Application Data
(63) Continuation of application No. 09/169,678, filed on Oct. 9, 1998.

(51) Int. Cl. A61K 31/4985

(52) U.S. Cl. 514/249

(57) ABSTRACT

A method for inhibiting neoplasia, particularly cancerous and precancerous lesions by exposing the affected cells to pyrido[3,4b]indoles.

### TECHNICAL FIELD

This invention relates to a method for the selective inhibition of neoplastic cells, for example, for the treatment or prevention of precancerous lesions or other neoplasias in mammals. This application is a Continuation of prior U.S. application Ser. No. 09/169,678, filed Oct. 9, 1998 entitled “Method of Inhibiting Neoplastic Cells with Tetracyclic Pyrido[3,4-B] Indole Derivatives” which is a Continuation-in-Part of U.S. patent application Ser. No. 09/007,098, filed Jan. 14, 1998 entitled “Method of Inhibiting Neoplastic Cells with Tetracyclic Pyrido[3,4-B] Indole Derivatives” both of which are incorporated herein by reference.

### BACKGROUND OF THE INVENTION

Each year in the United States alone, untold numbers of people develop precancerous lesions, which is a form of neoplasia, as discussed below. Such lesions exhibit a strong tendency to develop into malignant tumors, or cancer. Such lesions include lesions of the breast (that can develop into breast cancer), lesions of the skin (that can develop into malignant melanoma or basal cell carcinoma), colonic adenomatous polyps (that can develop into colon cancer), and other such neoplasms. Compounds that prevent or induce the remission of existing precancerous or cancerous lesions or carcinomas would greatly reduce illness and death from cancer.

For example, approximately 60,000 people die from colon cancer, and over 150,000 new cases of colon cancer are diagnosed each year. For the American population as a whole, individuals have a six percent lifetime risk of developing colon cancer, making it the second most prevalent form of cancer in the country. Colon cancer is also prevalent in Western Europe. It is believed that increased dietary fat consumption is increasing the risk of colon cancer in Japan.

In addition, the incidence of colon cancer reportedly increases with age, particularly after the age of 40. Since the mean ages of populations in America and Western Europe are increasing, the prevalence of colorectal cancer should increase in the future.

To date, little progress has been made in the prevention and treatment of colorectal cancer, as reflected by the lack of change in the five-year survival rate over the last few decades. The only cure for this cancer is surgery at an extremely early stage. Unfortunately, most of these cancers are discovered too late for surgical cure. In many cases, the patient does not experience symptoms until the cancer has progressed to a malignant stage.

In view of these grim statistics, efforts in recent years have concentrated on colon cancer prevention. Colon cancer usually arises from pre-existing benign neoplastic growths known as polyps. Prevention efforts have emphasized the identification and removal of colonic polyps. Polyps are identified by x-ray and/or colonoscopy, and usually removed by devices associated with the colonoscope. The increased use of colon x-rays and colonoscopies in recent years has detected clinically significant precancerous polyps in four to six times the number of individuals per year that acquire colon cancer. During the past five years alone, an estimated 3.5 to 5.5 million people in the United States have been diagnosed with adenomatous colonic polyps, and it is estimated that many more people have or are susceptible to developing this condition, but are as yet undiagnosed. In fact, there are estimates that 10-12 percent of people over the age of 40 will form clinically significant adenomatous polyps.

Removal of polyps has been accomplished either with surgery or fiber-optic endoscopic polypectomy—procedures that are uncomfortable, costly (the cost of a single polypectomy ranges between $1,000 and $1,500 for endoscopic treatment and more for surgery), and involve a small but significant risk of colon perforation. Overall, about $2.5 billion is spent annually in the United States in colon cancer treatment and prevention.

In the breast, breast cancer is often treated surgically, often by radical mastectomy with its painful aftermath. Such surgery is costly, too.

As indicated above, each lesion carries with it a chance that it will develop into a cancer. The likelihood of cancer is diminished if a precancerous lesion is removed. However, many of these patients demonstrate a propensity for developing additional lesions in the future. They must, therefore, be monitored periodically for the rest of their lives for reoccurrence.

In most cases (i.e. the cases of sporadic lesion formation, e.g. so-called common sporadic polyps), lesion removal will be effective to reduce the risk of cancer. In a small percentage of cases (i.e. cases where numerous lesions form, e.g. the so-called polyposis syndromes), removal of all or part of the effected area (e.g. the colon) is indicated. For example, the difference between common sporadic polyps and polyposis syndromes is dramatic. Common sporadic polyp cases are characterized by relatively few polyps which can usually be removed leaving the colon intact. By contrast, polyposis syndrome cases can be characterized by many (e.g. hundreds or more) of polyps—literally covering the colon in some cases—making safe removal of the polyps impossible short of surgical removal of the colon.

Because each lesion carries with it a palpable risk of cancerous development, patients who form many lesions (e.g. polyposis syndrome patients) invariably develop cancer if left untreated. Surgical removal of the colon is the conventional treatment in polyposis patients. Many polyposis patients have undergone a severe change in lifestyle as a result of the disfiguring surgery. Patients have strict dietary restrictions, and many must wear ostomy appliances to collect their intestinal wastes.

The search for drugs useful for treating and preventing cancer is intensive. Indeed, much of the focus of cancer research today is on the prevention of cancer because chemotherapy for cancer itself is often not effective and has severe side effects. Cancer chemoprevention is important for recovered cancer patients who retain a risk of cancer reoccurrence. Also, cancer prevention is important for people who have not yet had cancer, but have hereditary factors that place them at risk of developing cancer. With the development of new genetic screening technologies, it is easier to identify those patients with high-risk genetic factors, such as the potential for polyposis syndrome, who would greatly benefit from chemopreventative drugs. Therefore, finding such anti-cancer drugs that can be used for prolonged preventive use is of vital interest.

Known chemopreventative and chemotherapeutic drugs are believed to kill cancer cells by inducing apoptosis, sometimes referred to as “programmed cell death.” Apoptosis naturally occurs in virtually all tissues of the body, and especially in self-renewing tissues such as bone marrow, immune cells, gut, liver and skin. Apoptosis plays a critical role in tissue homeostasis, that is, it ensures that the number of new cells produced are correspondingly offset by an equal number of cells that die. For example, the cells in the intestinal lining divide so rapidly that the body must eliminate cells after only three days in order to prevent the overgrowth of the intestinal lining.

Recently, scientists have realized that abnormalities of apoptosis can lead to the formation of precancerous lesions and carcinomas. Also, recent research indicates that defects in apoptosis play a major role in other diseases in addition to cancer. Consequently, compounds that modulate apoptosis could be used to prevent or control cancer, as well as used in the treatment of other diseases.

Unfortunately, even though known chemotherapeutic drugs may exhibit such desirable apoptosis effects, most chemotherapeutic drugs have serious side effects that prohibit their long-term use, or use in otherwise healthy individuals with precancerous lesions. These side effects, which are a result of the high levels of cytotoxicity of the drugs, include hair loss, weight loss, vomiting, immune suppression and other toxicities. Therefore, there is a need to identify new drug candidates for therapy that do not have such serious side effects in humans.

In recent years, several non-steroidal anti-inflammatory drugs (“NSAIDs”), originally developed to treat arthritis, have shown effectiveness in inhibiting and eliminating colonic polyps. Polyps virtually disappear when the patients take the drug, particularly when the NSAID sulindac is administered. However, the prophylactic use of currently available NSAIDs, even in polyposis syndrome patients, is marked by severe side reactions that include gastrointestinal irritations, perforations, ulcerations and kidney toxicity. Once NSAID treatment is terminated due to such complications, the polyps return, particularly in polyposis syndrome patients.

Sulindac has been particularly well received among the NSAIDs for polyp treatment. Sulindac is a suboxide compound that itself is believed to be inactive as an anti-arthritic agent. The sulfoxide is reportedly converted by liver enzymes to the corresponding sulfide, which is acknowledged to be the active moiety as a prostaglandin synthesis inhibitor. The sulfide, however, is associated with the side effects of conventional NSAIJDs. The sulfoxide is also known to be metabolized to a sulfone compound that has been found to be inactive as an inhibitor of prostaglandin synthesis but active as an inhibitor of precancerous lesions.

### SUMMARY OF THE INVENTION

This invention includes a method of inhibiting neoplastic cells by exposing those cells to a pharmacologically effective amount of those compounds described below. Such compounds are effective in modulating apoptosis and eliminating and inhibiting the growth of neoplasias such as precancerous lesions, but are not characterized by the severe side reactions of conventional NSAJDs or other chemotherapeutics.

The compounds of that are useful in the methods of this invention include those of Formula I:

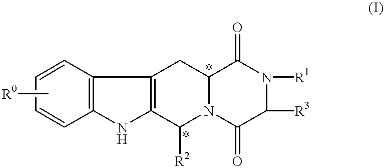

wherein R⁰ is selected from the group consisting of hydrogen, halogen or C₁₋₆ alkyl;

R¹ is selected from the group consisting of hydrogen, C₁₋₆ alkyl, C₂₋₆ alkenyl, C₂₋₆ alkynyl, haloC₁₋₆alkyl, C₃₋₈cycloalkyl, C₃₋₈cycloalkyl C₁₋₃alkyl, arylC₁₋₃alkyl or heteroarylC₁₋₃alkyl;

R² is an optionally substituted monocyclic aromatic ring selected from benzene, thiophene, furan and pyridine or an optionally substituted bicyclic ring

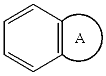

attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from the group consisting of oxygen, sulphur and nitrogen; and

R³ is selected from the group consisting of hydrogen or C₁₋₃ alkyl, or R¹ and R³ together represent a 3- or 4-membered alkyl or alkenyl chain.

### DETAILED DESCRIPTION OF THE PREFERRED EMBODIMENTS

As indicated above, this invention relates to a method for inhibiting neoplasia, particularly cancerous and precancerous lesions by exposing the affected cells to a compound of Formula I above. Preferred compounds useful in the practice of this invention include those wherein R¹ is selected from the group consisting of hydrogen, C¹⁻⁶alkyl, haloC₁₋₆alkyl, C₃₋₈cycloalkyl, C₃₋₈cycloalkylC₁₋₃alkyl, arylC₁₋₃alkyl or heteroarylC₁₋₃alkyl; and

Within R¹ above, the term “aryl” as part of an arylC₁₋₃ alkyl group means phenyl or phenyl substituted by one or more (e.g. 1, 2 or 3) substituents selected from halogen, C₁₋₆alkyl, C₁₋₆alkoxy and methylenedioxy. The term “heteroaryl” as part of a heteroarylC₁₋₃alkyl group means thienyl, furyl or pyridyl each optionally substituted by one or more (e.g. 1, 2 or 3) substituents independently selected from halogen, C₁₋₆alkyl and C₁₋₆alkoxy. The term “C₃₋₈cycloalkyl” as a group or part of a C₃₋₈cycloalkylC₁₋₃alkyl group means a monocyclic ring comprising three to eight carbon atoms. Examples of suitable cycloalkyl rings include the C3-6cydoalkyl rings cyclopropyl, cyclobutyl, cyclopentyl and cyclohexyl.

Within R² above, optional benzene ring substituents are selected from one or more (e.g. 1, 2 or 3) atoms or groups comprising halogen, hydroxy, C₁₋₆alkyl, C₁₋₆alkoxy, —CO₂R_{b}, haloC₁₋₆alkyl, haloC₁₋₆alkoxy, cyano, nitro and NRₐR_{b}, where Rₐ and R_{b} are each hydrogen or C₁₋₆alkyl, or Rₐ may also represent C₂₋₇alkanoyl or C₁₋₆alkylsulphonyl.

Optional substituents for the remaining ring systems are selected from one or more (e.g. 1, 2 or 3) atoms or groups comprising halogen, C₁₋₆alkyl, C₁₋₆alkoxy and arylC₁₋₃alkyl as defined above. The bicyclic ring

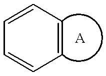

may, for example, represent naphthalene, a heterocycle such as benzoxazole, benzothiazole, benzisoxazole, benziridazole. quinoline, indole, benzothiophene or benzoiran or

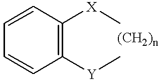

(where n is an integer 1 or 2 and X and Y may each represent CH₂, O, S or NH.

In the above definitions, the term “alkyl” as a group or part of a group means a straight chain or, where available, a branched chain alkyl moiety. For example, it may represent a C₁₋₄alkyl function as represented by methyl, ethyl, n-propyl, i-propyl, n-butyl, s-butyl and t-butyl. The term ‘alkenyl’ as used herein includes straight-chained and branched alkenyl groups, such as vinyl and allyl groups. The term ‘alkynyl’ as used herein includes straight-chained and branched alkynyl groups, suitably acetylene. The term “halogen” herein means a fluorine, chlorine, bromine or iodine atom. The term “haloC₁₋₆alkyl” means an alkyl group as defined above comprising one to six carbon atoms substituted at one or more carbon atoms by one or more (e.g. 1, 2 or 3) halogen atoms. Similarly, a haloC₁₋₆alkoxy group is a haloC₁₋₆alkyl group as defined above linked to the R₂ benzene ring via an oxygen atom. Examples of haloC₁₋₆alkyl groups include trifluoromethyl and 2,2,2-trifluoroethyl. An example of a haloC₁₋₆alkoxy group is trifluoromethoxy. The term “C₂₋₇alkanoyl” means a C₁₋₆alkylcarbonyl group where the C₁₋₆))alkyl portion is as defined above. An example of a suitable C₂₋₇alkanoyl group is the C₂alkanoyl group acetyl.

It will be appreciated that when R₀ is a halogen atom or a C₁₋₆alkyl group this substituent may be sited at any available position on the phenyl portion of the tetracyclic ring. However, a particular site of attachment is the ring 10-position.

The compounds of Formula I may contain two or more asymmetric centres and thus can exist as enantiomers or diastereoisomers. In particular, in Formula I above two ring chiral centers are denoted with asterisks. It is to be understood that the compounds useful in the practice of this invention include both mixtures and separate individual isomers of the compounds of Formula I.

The compounds of Formula I may also exist in tautomeric forms, and the practice of this invention can include both mixtures and separate individual tautomers thereof.

The pharmaceutically acceptable salts of the compounds of Formula I which contain a basic center are acid addition salts formed with pharmaceutically acceptable acids. Examples include the hydrochloride, hydrobromide, sulphate or bisulphate, phosphate or hydrogen phosphate, acetate, benzoate, succinate, fumarate, maleate, lactate, citrate, tartrate, gluconate, methanesulphonate, benzenesulphonate and p-toluenesulphonate salts. Compounds of Formula I can also provide pharmaceutically acceptable metal salts, in particular alkali metal salts, with bases. Examples include the sodium and potassium salts.

A particular group of compounds useful in the practice of the invention are those compounds of Formula I wherein

R⁰ is hydrogen or halogen (e.g., fluorine), especially hydrogen.

Another preferred group of compounds useful in this practice of the invention are those compounds of Formula I wherein R¹ is selected from the group consisting of hydrogen, C₁₋₄alkyl, haloC₁₋₄alky, C₃₋₆cycloalyl, C₃₋₆cycloalklylmethyl, pyridylC₁₋₃allyl, furylC₁₋₃alkyl or optionally substituted benzyl. Within this particular group of compounds, examples of C₁₋₄ alkyl groups are methyl, ethyl, n-propyl, i-propyl and n-butyl. Examples of C₃₋₆cycloalkylmethyl groups are cyclopropylmethyl and cyclohexylmethyl. Examples of optionally substituted, benzyl groups include benzyl and halobenzyl (e.g., fluorobenzyl).

A further particular group of compounds useful in the practice of the invention are those compounds of Formula I wherein R² is selected from the group consisting of an optionally substituted benzene, thiophene,furan, pvridine or naphthalene ring or an optionally substituted bicyclic ring

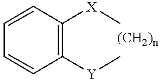

where n is 1 or 2 and X and Y are each CH₂ or O. Within this particular group of compounds, examples of substituted benzene groups are benzene substituted by one of halogen (e.g. chlorine), hydroxy, C₁₋₃alkyl (e.g., methyl, ethyl or i-propyl), C₁₋₃alkoxy (e.g. methoxy or ethoxy), —CO₂R^{b}, halomethyl (e.g., trifluoromethyl), halomethoxy (e.g., trifluoromethoxy), cyano, nitro or NR^{a}R^{b} where R^{a} and R^{b} are each hydrogen or methyl or Rₐ is acetyl; or benzene substituted by dihalo (e.g., dichloro) or by C₁₋₃alkoxy (e.g., methoxy) and one of halogen (e.g., chlorine) and hydroxy. Preferably, R² is a is benzofuran, attached to the tetracyclic structure at the 5-position of the benzofuran moiety.

An example of a substituted thiophene ring is a halo (e.g. bromo) substituent thiophene ring.

A still further particular group of compounds of Formula I are those wherein R₃ is hydrogen or or methyl, or R¹ and R³ together represent a 3 -membered alkyl chain.

A preferred group of compounds useful in the practice of the invention are the cis isomers of Formula I represeted by Formula Ib

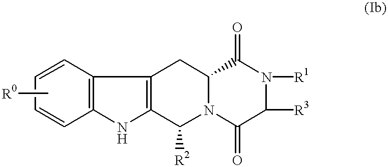

and mixtures thereof with their cis optical enantiomers, including racemic mixtures, and salts and solvates (e.g., hydrates) of these compounds in which R₀ is hydrogen or halogen (e.g., fluorine), especially hydrogen and R¹, R² and R³ are as defined previously.

The single isomers represented by Formula Ib, i.e., the 6R, 12aR isomers, are particularly preferred.

Within the above definitions R¹ may preferably represent C₁₋₄alkyl (e.g., methyl, ethyl, i-propyl and n-butyl), C₃₋₆cycloalkyl (e.g., cyclopentyl) or C₃₋₆cycloalkylmethyl (e.g., cyclopropylmethyl).

R² may preferably represent a substituted benzene ring such as benzene substituted by C₁₋₃alkoxy (e.g., methoxy) or by C₁₋₃alkoxy (e.g., methoxy) and halogen (e.g., chlorine), particularly 4-methoxyphenyl or 3-chloro-4-methoxyphenyl, or R² may preferably represent 3,4-methylenedioxyphenyl.

It is to be understood that the present invention involves the use of all appropriate combinations of particular and preferred groupings hereinabove.

Particular individual compounds useful in the practice of the invention include:

Cis-2,3,6,7,12,12a-hexahydro-2-(4-pyridylmethyl)-6-(3,4-methylenedioxyphenyl)-pyrazino(2′,1′:6,1)pyrido(3,4-b)indole-1,4-dione;

Cis-2,3,6,7,12,12a-hexahydro-6-(2,3-dihydrobenzo(b)furan-5-yl)-2-methylpyrazino(2′,1′:6,1)pyrido(3,4-b)indole-1,4-dione;

Cis-2,3,6,7,12,12a-hexahydro-6-(5-bromo-2-thienyl)-2-methyl-pyrazino(2′,1′:6,1)pyrido(3,4-b)indole-1,4-dione;

Cis-2,3,6,7,12,12a-hexahydro-2-butyl-6-(4-methylphenyl)pyrazino(2′,1′:6,1)pyrido(3,4-b)indole- 1,4-dione;

(6R,12aR)-2,3,6,7,2,12a-Hexahydro-2-isopropyl-6-(3,4-methylenedioxyphenyl)-pyrazino(2′,1′:6,1 )pyrido(3,4-b)indole-1,4-dione;

(6R,12aR)-2,3,6,7,12,12a-Hexahydro-2-cyclopentyl-6-(3,4-methylenedioxyphenyl)-pyrazino(2′,1′:6,1)pyrido(3,4-b)indole-1,4-dione;

(6R,12aR)-2,3,6,7,12,12a-Hexahydro-2-cyclopropylmethyl-6-(4-methoxyphenyl)pyrazino(2′,1′:6,1)pyrido(3,4-b)indole-1,4-dione;

(6R,12aR)-2,3,6,7,12,12a-Hexahydro-6-(3-chloro-4-methoxyphenyl)-2-methyl-pyrazino(2′,1′:6,1)pyrido(3,4-b)indole-1,4dione;

(6R,12aR)-2,3,6,7,12,12a-Hexahydro-2-methyl-6-(3,4-methylenedioxyphenyl)pyrazino(2′,1′:6,1)pyrido(3,4-b)indole-1,4-dione;

(6R,12aR)-2,3,6,7,12,12a-Hexahydro-6-(3,4-methylenedioxyphenyl)pyrazino(2′,1′:6,1)pyrido(3,4-b) indole-1,4-dione;

(5aR, 12R, 14aS)-1,2,3,5,6,11,12,14a-Octahydro-12-(3,4-methylenedioxyphenyl)-pyrrolo(1″,2″:4′,5′)pyrazino(2′,1′:6,1)pyrido(3,4-b)indole-5-1,4-dione;

(6R, 12aR)-2,3,6,7,12,12a-hexahydro-6-(5-benzofuranyl)-2-methyl-pyrazino [2′, 1′:6,1] pyrido[3,4-b]indole-1,4-dione;

(6R, 12aR)-2,3,6,7,12,12a-hexahydro-6-(5-benzofuranyl)-pyrazino[2′, 1′:6,1] pyrido [3,4-b]indole-1,4-dione;

(3S, 6R, 12aR)-2,3,6,7,12,12a-hexahydro-6-(5-benzofuranyl)-3-methylpyrazino[2′,1′-6,1] pyrido [3,4-b]indole-1,4-dione;

(3S, 6R, 12aR)-2,3,6,7,12,12a-hexahydro-6-(5-benzofuranyl)-2,3-dimethylpyrazino[2′,1′:6,1] pyrido [3,4-b]indole-1,4-dione;

(6R, 12aR)-2,3,6,7,12,12a-hexahydro-6-(5-benzofuranyl)-2-isopropyl-pyrazino [2′,1′:6,1] pyrido [3,4-b]indole-1,4-dione;

and physiologically acceptable solvates (e.g. hydrates) thereof

Specifically preferred compounds useful in the practice of the invention are: (6R, 12aR)-2,3,6,7,12,12a-hexahydro-6-(5 -benzofuranyl)-2-methyl-pyrazino [2′,1′:6,1]pyrido[3,4-b]indole-1,4-dione and (6R,12aR)-2,3,6,7,12,12a-hexahydro-2-methyl-6-(3,4-methylenedioxyphenyl)pyrazino(2′,1′:6,1)pyrido(3,4-b)indole-1,4-dione; and physiologically acceptable salts and solvates (e.g., hydrates) thereof.

Preferably, such compounds are administered without therapeutic amounts of an NSAID.

The present invention is also a method of treating mammals with precancerous lesions by administering a pharmacologically effective amount of an enterically coated pharmaceutical composition that includes compounds of this invention.

Also, the present invention is a method of inhibiting the growth of neoplastic cells by exposing the cells to an effective amount of compounds of Formula I, wherein R₁ through R₃ are defined as above.

In still another form, the invention is a method of inducing apoptosis in human cells by exposing those cells to an effective amount of compounds of Formula I to those cells sensitive to such a compound.

As used herein, the term “precancerous lesion” includes syndromes represented by abnormal neoplastic, including dysplastic, changes of tissue.

Examples include adenomatous growths in colonic, breast or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

As used herein, the term “carcinomas” refers to lesions that are cancerous. Examples include malignant melanomas, breast cancer, and colon cancer.

As used herein, the term “neoplasm” refers to both precancerous and cancerous lesions.

It will also be appreciated that a compound of Formula I or a physiologically acceptable salt or solvate thereof can be administered as the raw compound, or as a pharmaceutical composition containing either entity.

Compounds useful in the methods of this invention are preferably formulated into compositions together with pharmaceutically acceptable carriers for oral administration in solid or liquid form, or for rectal administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g. pharmaceutically acceptable emulsions, solutions, suspensions, syrups and elixirs containing inert diluents commonly used in the art, such as water. Besides such inert diluents, compositions can also include adjuvants such as wetting agents, emulsifyg and suspending agents, and sweetening, flavoring and perfuming agents.

Pharmaceutically acceptable carriers for rectal administration are preferably suppositories that may contain, in addition to the compounds of Formula I, excipients such as cocoa butter or a suppository wax.

The pharmaceutically acceptable carrier and compounds of this invention are formulated into unit dosage forms for administration to a patient. The dosage levels of active ingredient (i.e. compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The pharmaceutical compositions of this invention are preferably packaged in a container (e.g. a box or bottle, or both) with suitable printed material (e.g. a package insert) containing indications, directions for use, etc.

For administration to humans in the curative or prophylactic treatment of the disorders identified above, oral dosages of a compound of Formula I will generally be in the range of from 0.5-800 mg daily for an average adult patient (70 kg). Thus for a typical adult patient, individual tablets or capsules contain from 0.2-400 mg of active compound, in a suitable pharmaceutically acceptable vehicle or carrier, for administration in single or multiple doses, once or several times per day. Dosages for intravenous, buccal or sublingual administration will typically be within the range of from 0.1- 400 mg per single dose as required. In practice, the physician will determine the actual dosing regimen that will be most suitable for an individual patient and it will vary with the age, weight and response of the particular patient. The above dosages are believed to be exemplary of the average case, but there may be individual instances in which higher or lower dosage ranges may be merited, and such are within the scope of this invention.

Compounds of Formula I may be prepared by any suitable method known in the art or by the following processes disclosed in WO95/19978 and WO97/03985. In the methods below R₀, R₁ and R₂ are as defined in Formula I above unless otherwise. indicated.

A process (A) for preparing a compound of Formula I wherein R₃ is hydrogen comprises treating a compound of Formula II

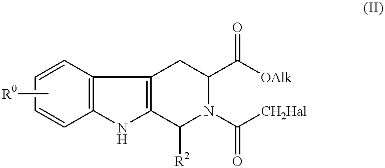

(in which Alk represents C₁₋₆alky, e.g., methyl or ethyl and Hal is a halogen atom, e.g., chlorine) with a primary amine R¹NH₂ in a suitable solvent such as an alcohol (e.g., methanol or ethanol) or a mixture of solvents, conveniently at a temperature of from 20° C. to reflux (e.g., at about 50° C.).

A compound of Formula II may conveniently be prepared by treating a compound of Formula III

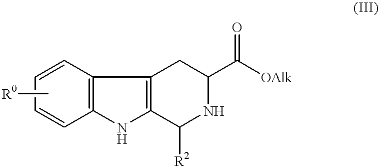

with a haloacetyl halide (e.g., chloroacetyl chloride) in a suitable solvent such as a halogenated hydrocarbon (e.g., trichloromethane or dichloromethane), or an ether (e.g., tetrahydrofuran), preferably in the presence of a base such as an organic amine (e.g., a trialkylamine such as triethylamine) or an alkali metal carbonate or bicarbonate (e.g., NaHCO₃). The reaction may conveniently be effected at a temperature of from −20° C. to +20° C. (e.g. at about 0° C.).

A compound of Formula I may also be prepared from a compound of Formula III in a two-step procedure via a compound of Formula II isolated without purification.

Compounds of Formula I may be prepared as individual enantiomers in two-steps from the appropriate enantiomer of Formula III or as mixtures (e.g., racemates) of either pairs of cis or trans isomers from the corresponding mixtures of either pairs of cis or trans isomers of Formula III.

Individual enantiomers of the compounds usefull in the practice of the invention may be prepared from racemates by resolution using methods known in the art for the separation of racemic mixtres into their constituent enantiomers, for example using HPLC (high performance liquid chromatography) on a chiral column such as Hypersil naphthylurea.

A compound of Formula III reportedly may conveniently be prepared from a tryptophan alkyl ester of Formula IV

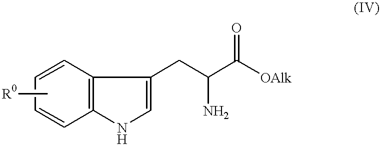

(where Alk is as previously defined) or a salt thereof (e.g. the hydrochloride salt) according to either of the following procedures (a) and (b). Procedure (b) is only suitable for preparing cis isomers of Formula III and may be particularly suitable for preparing individual cis enantiomers of Formula III from D- or L-tryptophan alkyl esters as appropriate.

Procedure (a)

This comprises a Pictet-Spengler cyclisation between a compound of Formula IV and an aldehyde R₂CHO. The reaction may conveniently be effected in a suitable solvent such as a halogenated hydrocarbon (e.g., dichloromethane) or an aromatic hydrocarbon (e.g. toluene) in the presence of an acid such as trifluoroacetic acid. The reaction may conveniently be carried out at a temperature of from −20° C. to reflux to provide a compound of Formula III in one step. The reaction may also be carried out in a solvent such as an aromatic hydrocarbon (e.g., benzene or toluene) under reflux, optionally using a Dean-Stark apparatus to trap the water produced.

The reaction provides a mixture of cis and trans isomers which may be either individual enantiomers or racemates of pairs of cis or trans isomers depending upon whether racemic or enantiomerically pure tryptophan alkyl ester was used as the starting material. Individual cis or trans enantomers may conveniently be separated from mixtures thereof by fractional crystallisation or by chromatography (e.g., flash column chromatography) using appropriate solvents and eluents. Similarly, pairs of cis and trans isomers may be separated by chromatography (e.g., flash column chromatography) using appropriate eluents. An optically pure trans isomer may also be converted to an optically pure cis isomer using suitable epimerisation procedures. One such procedure comprises treating the trans isomer or a mixture (e.g., 1:1 mixture) of cis and trans isomers with methanolic or aqueous hydrogen chloride at a temperature of from 0° C. to the refluxing temperature of the solution. The mixture may then be subjected to chromatography (e.g., flash column chromatography) to separate the resulting diastereoisomers, or in the procedure utilising aqueous hydrogen chloride the desired cis isomer precipitates out as the hydrochloride salt which may then be isolated by filtration.

Procedure (b)

This comprises a four-step procedure from a compound of Formula IV or a salt thereof (e.g., the hydrochloride salt). The procedure is particularly suitable for preparing a 1R, 3R isomer of Formula III from a D-tryptophan alkyl ester of Formula IV or a salt thereof (e.g., the hydrochloride salt). Thus, a first step (i) comprises treating a compound of Formula IV with an acid halide R₂COHal (where Hal is as previously defined) in the presence of a base, e.g., an organic base such as a trialkylamine (for example, triethylamine), to provide a compound of Formula V

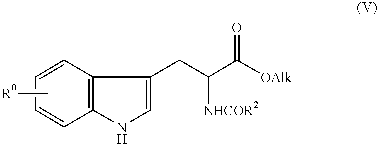

The reaction may be conveniently carried out in a suitable solvent such as a halogenated hydrocarbon (e.g., dichloromethane) or an ether (e.g., tetrahydrofuran) and at a temperature of from −20° C. to +40° C.

Step (ii) comprises treating a compound of Formula V with an agent to convert the amide group to a thioamide group. Suitable sulfurating agents are well-known in the art. Thus, for example, the reaction may conveniently be effected by treating (V) with Lawesson's reagent. This reaction may conveniently be carried out in a suitable solvent such as an ether (e.g., dimethoxyethane) or an aromatic hydrocarbon (e.g., toluene) at an elevated temperature such as from 40° C. to 80° C. to provide a compound of Formula VI

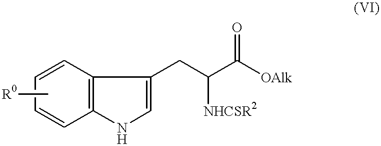

Step (iii) comprises treating a compound of Formula VI with a suitable agent to provide a compound of Formula VII

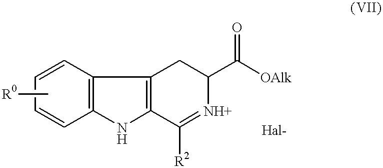

(where Hal is a halogen atom, e.g., iodine). The reaction may conveniently be effected by treating VI with an alkylating agent such as a methyl halide (e.g., methyl iodide) or an acylating agent such as an acetyl halide (e.g., acetyl chloride) in a suitable solvent such as a halogenated hydrocarbon (e.g., dichloromethane) at an elevated temperature (e.g., under reflux).

In step (iv) the resulting iminium halide of Formula VII may be treated with a reducing agent such as boron hydride, e.g., sodium borohydride, to provide the desired compound of Formula III. The reduction may conveniently be effected at a low temperature, e.g. within the range of −100° C. to 0° C., in a suitable solvent such as an alcohol (e.g. methanol).

A process (B) for preparing a compound of Formula I, wherein R¹ and R³ together represent a 3- or 4-membered alkyl or alkenyl chain, which process (B) comprises cyclization of a compound of Formula VIII

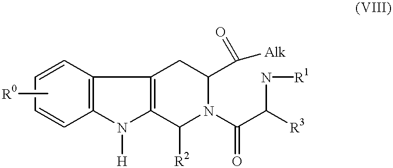

wherein Alk represents C₁₋₆alkyl and R¹ and R³ together represent a 3- or 4-membered chain both as described previously. The cyclization is suitably carried out in an organic solvent or solvents, such as an alcoholic solvent (e.g., methanol) and optionally an ether solvent such as tetrahydrofuran, and in the presence of a reducing agent, preferably a palladium catalyst, such as palladium on carbon.

Conveniently a compound of Formula VIII is prepared by reaction of a arm compound of Formula III as previously described with a compound of Formula IX

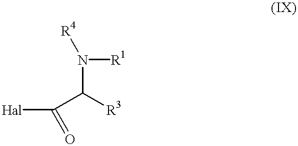

wherein Hal represents a halogen atom as hereinbefore described, R¹ and R³ together represent a 3- or 4-membered chain as hereinbefore described and R⁴ is a protecting group, suitably a benzyloxycarbonyl group or the like. Typically the reaction is carried out in a chlorinated organic solvent, such as dichloromethane, and a tertiary amine, such as triethylamine or the like.

A process (C) for preparing a compound of Formula I wherein R³ is C₁₋₃alkyl, which process comprises cyclization of a compound of Formula X

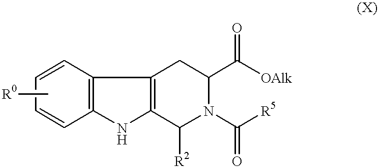

wherein Alk represents C₁₋₆alkyl as described previously and R₅ represents C₂₋₅ alkyl, substituted at Cl by a halogen atom, the halogen atom being as described above. Suitably the cyclization is achieved by reflux for many hours, such as 22 to 26 hours, in the presence of an ether solvent, such as tetrahydrofuran, and a suitable amine as hereinafter described in the accompanying examples.

A compound of Formula X can be prepared from a compound of Formula III by suitable acylation techniques, such as reaction with a C₃₋₆carboxylic acid, substituted at C₂ by a halogen atom in a halogenated organic solvent, such as dichloromethane.

Compounds of Formula I may be converted to other compounds of Formula I. For example, when R₂ is a substituted benzene ring, it may be necessary or desirable to prepare the suitably substituted compound of Formula I subsequent to process (A), (B) or (C) as above. Examples of appropriate interconversions include nitro to amino or aralkyloxy to hydroxy by suitable reducing means (e.g., using a reducing agent such as SnCl₂ or a palladium catalyst, such as palladium-on-carbon), or amino to substituted amino such as acylamino or sulphonylamino using standard acylating or sulphonylating conditions. In the case where R₂ represents a substituted bicyclic system, suitable interconversion can involve removal of a substituent, such as by treatment with a palladium catalyst (e.g. palladium-on-carbon) whereby, for example, a benzyl substituent may be removed from a suitable bicyclic system.

The pharmaceutically acceptable acid addition salts of the compounds of Formula I which contain a basic centre may be prepared in a conventional manner. For example, a solution of the free base may be treated with a suitable acid, either neat or in a suitable solution, and the resulting salt isolated either by filtration or by evaporation under vacuum of the reaction solvent.

Pharmaceutically acceptable base addition salts may be obtained in an analogous manner by treating a solution of a compound of Formula I with a suitable base. Both types of salt may be formed or interconverted using ionexchange resin techniques.

Compounds useful in this invention may be isolated in association with solvent molecules by crystallisation from or evaporation of an appropriate solvent. Thus, a process for preparing a compound of Formula I or a salt or solvate (e.g., hydrate) thereof which comprises process (A), (B) or (C) as hereinbefore described followed by

(i) an interconversion step; and/or either

(ii) salt formation; or

(iii) solvate (e.g., hydrate) formation.

The synthesis of the compounds of the invention and of the intermediates for use therein are illustrated by the following, non-limiting Examples from the aforesaid PCT applications. In the Examples section hereinafter the following abbreviations are used: DMSO (dimethylsulphoxide), MeOH (methanol), EtOH (ethanol), DMF (dimethylformamide), EtOAc (ethyl acetate) and TBF (tetrahydrofaran).

### INTERMEDIATES 1 AND 2

### Methyl 1,2,3,4-Tetrahydro-1-(3,4-Methylenedioxyphenyl)-9H-Pyrido(3,4-b)Indole-3-Carboxylate, Cis and Trans Isomers

To a stirred solution of racemic tryptophan methyl ester (13 g) and piperonal (9.7 g) in anhydrous CH₂Cl₂ (300 ml) cooled at 0° C. is added dropwise trifluoroacetic acid (9 ml), and the solution is allowed to react at ambient temperature. After 4 days, the yellow solution is diluted with CH₂Cl₂ (100 ml), washed with a saturated aqueous solution of NaHCO₃, then with water and dried over Na₂SO₄. The organic layer is evaporated to dryness under reduced pressure, and the residue is purified by flash chromatography eluting with CH₂Cl₂/MeOH (99/1) to give first Intermediate 1, the cis isomer, m.p. : 90-93° C. followed by Intermediate 2, the trans isomer (6.4 g) m.p.: 170° C.

The following compounds are obtained in a similar manner:

### INTERMEDIATES 3 AND 4

### Methyl 1,2,3,4-Tetrahydro-l-(4-Methoxyphenyl)-9H-Pyrido(3,4-b)Indole-3-Carboxylate, Cis and Trans Isomers

The same method but starting from racemic tryptophan methyl ester and 4-methoxybenzaldehyde gives Intermediate 3, the cis isomer as white crystals m.p.: 142° and Intermediate 4, the trans isomer as white crystals m.p.: 209-210° C.

### INTERiMEDIATE 5

### Methyl 1,2,3,4-Tetrahydro-1-(3-Methoxyphenyl)-9H-Pyrido(3,4-Blindole-3-Carboxylate, Cis Isomer

The same method but starting from racemic tryptophan methyl ester and 3-methoxybenzaldehyde givesthe title compound as white crystals m.p. 146° C.

### INTERMEDIATES 6 AND 7

### Methyl 1,2,3,4-Tetrahydro-1-(4-Ethoxyphenyl)-9H-Pyrido (3,4-b)Indole-3-Carboxylate,Cis and Trans Isomers

The same method but starting from racemic tryptophan methyl ester and 4-ethoxybenzaldehyde gives Intermediate 6, the cis isomer as white crystals m.p.: 180° C. and Intermediate 7, the trans isomer as white crystals m.p.: 196-198° C.

### INTERMEDIATES 8 AND 9

### Methyl 1,2,3,4-Tetrahydro-1-(2,3-Dihydrobenzo(b)Furan-5-yl)-9H-Pyrido(3,4-b)Indole-3-Carboxylate, Cis and Trans Isomers

The same method but starting from racemic tryptophan methyl ester and 2,3-dihydrobenzo(b)firan-5-carboxaldehyde gives Intermediate 8, the cis isomer as white crystals m.p.: 106-109° C. and Intermediate 9, the trans isomer as white crystals m.p.: 219-222° C.

### INTERMEDIATES 10 AND 11

### Methyl 1,2,3,4-Tetrahydro-1-(3,4-Ethylenedioxyphenyl)-9H-Pyrido(3,4-b)Indole-3-Carboxylate, Cis and Trans Isomers

The same method but starting from racemic tryptophan methyl ester and 1,4-benzodioxan-6-carboxaldehyde gives Intermediate 10, the cis isomer as white crystals m.p.: 104-106° C. and Intermediate 11, the trans isomer as white crystals m.p.: 207-2090C.−

### INTERMEDIATE 12

### Methyl 1,2,3,4-Tetrahydro-1-(2-Chlorophenyl)-9H-Pyrido(3,4-b)Indole-3-Carboxylate,Mixture of Cis and Trans Isomers

The same method but starting from racemic tryptophan methyl ester and 2-chlorobenzaldehyde gives the title compound as white crystals m.p.: 154° C.

### INTERMEDIATES 13 ALND 14

### Methyl 1,2,3,4-Tetrahydro-1-(4-Chlorophenyl)-9H-Pyrido (3,4-b)Indole-3-Carboxylate Cis and Trans Isomers

The same method but starting from racemic tryptophan methyl ester and 4-chlorobenzaldehyde gives Intermediate 13, the cis isomer as white crystals m.p. 208-209° C. and Intermediate 14, the trans isomer as white crystals m.p.: 108-109° C.

### INTERMEDIATES 15 AND 16

### Methyl 1,2,3,4-Tetrahydro-l-(3,4-Dichlorophenyl)-9H-Pyrido (3,4-b)Indole-3-Carboxylate, Cis and Trans Isomers

The same method but starting from racemic tryptophan methyl ester and 3,4-dichlorobenzaldehyde gives Intermediate 15, the cis isomer as a white solid ¹H NMN (CDCl₃) (delta) (ppm) 7.8-7 (m, 8H, H aromatic); 5.15 (brs, 1H, H-1); 3.9-3.8 (dd, 1H, H-3) 3.7 (s, 3H, CO₂CH₃) ; 3.2-3.1 (ddd, 1H, H-4) 2.9 (m, 1H, H-4) 2.4 (brs,1H, NH) and Intermediate 16, the trans isomer as a white solid m.p.: 204° C.

### INTERMEDIATE 17

### Methyl 1,2,3,4-Tetrahydro-1-(1,2,3,4-Tetrahydro-6-Naphthyl)-9H-Pyrido(3-4b)Indole-3-Carboxylate, Cis Isomer

The same method but starting from racemic tryptophan methyl ester and 1,2,3,4-tetrahydronaphthyl-6-carboxaldehyde gives the title compound as a white solid ¹HNMJ (CDCl₃) (delta) (pm): 7.7-7(m, 8H, H aromatic) ; 5.2 (s, 1H, H-1); 4.0 (dd, 1H, H-3) ; 3.8 (s, 3H, CO₂CH₃) ; 3.2 (m, 1H, H-4) ; 3.0 (m, 1H, H-4) ; 2.7 (m, 4H, CH₂Ar); 1.7 (s, 4H, CH₂CH₂Ar).

### INTERIEDIATES 18 A-ND 19

### Methyl 1,2,3,4-Tetrahydro-1-(2-Naphthyl)-9H-Pyrido (3,4-b)Indole-3-Carboxylate, Cis and Trans Isomers

The same method but starting from racemic typtophan methyl ester and 2-naphthaldehyde gives Intermediate 18, the cis isomer as a white solid ¹H NMR (CDCl₃) (delta) (ppm): 8-6.9 (m, 12H, H aromatic) ; 5.4 (s, 1H, H-1) ; 3.95 (dd, 1H, H-3) ; 3.7 (s, 3H, CO₂CH₃) 3.2 (ddd, 1H, H-4) ; 3 (m, 1H, H-4) ; 2.5 (brs, 1H, NH) and Intermediate 19, the trans isomer as a white solid, m.p.: 119° C.

### INTERMEDIATES 20 AND 21

### Methyl 1,2,3,4-Tetrahydro-1-(2-Thienyl)-9H-Pyrido (3,4-b)Indole-3-Carboxylate Cis and Trans Isomers

The same method but starting from racemic tryptophan methyl ester and 2-thiophenecarboxaldehyde gives Intermediate 20, the cis isomer as a pale yellow solid m.p.: 134-137° C. and Intermediate 21, the trans isomer as white crystals m.p.: 169° C.

### INTERMEDIATES 22 AND 23

### Ethyl 1,2,3,4-Tetrahydro-1 -(3 -Thienyl)-9H-Pyrido(3,4-b) Indole-3-Carboxylate, Cis and Trans Isomers

The same method but starting from racemic tryptophan ethyl ester and 3-thiophenecarboxaldehyde gives Intermediate 22, the cis isomer as white crystals m.p.: 130° C. and Intermediate 23, the trans isomer as white crystals m.p.: 182-184° C.

### INTERMEDIATES 24 AND 25

### Methyl 1,2,3,4-Tetrahydro-1-(5-Bromo-2-Thienyl)-9H-Pyrido (3 4-b)Indole-3-Carboxylate, Cis and Trans Isomers

The same method but starting from racemic tryptophan methyl ester and 5-bromo-2-thiophenecarboxaldehyde gives Intermediate 24, the cis isomer as a cream solid m.p.: 130° C. and Intermediate 25, the trans isomer as a cream solid m.p.: 205° C.

### INIERMEDIATES 26 AND 27

### Methyl 1,2,3,4-Tetrahydro-1-(4-Bromo-2-Thieny))-9H-Pyrido (3,4-b)Indole-3-Carboxylate. Cis and Trans Isomers

The same method but starting from racemic tryptophan methyl ester and 4-bromo-2-thiophenecarboxaldehyde gives Intermediate 26, the cis isomer as a cream solid m.p.: 200° C. and Intermediate 27, the trans isomer as a cream solid m.p.: 120° C.

### INTERMEDIATE 28

### Methyl 1,2,3,4-Tetrahydro-1-(3-Furyl)-9H-Pyrido(3,4-b) Indole-3-Carboxylate. Mixture of Cis and Trans Isomers

The same method but starting from racemic tryptophan methyl ester and 3-furaldehyde gives the title compound as a yellow solid m.p.: 130° C.

### INTERMEDIATES 29 AND 30

### Ethyl 1,2,3,4-Tetrahydro-1-(5-Methyl-2-Furyl)-9H-Pyrido (3,4-b)Indole-3-Carboxylate. Cis and Trans Isomers

The same method but starting from racemic tryptophan ethyl ester and 5-methylfurfuiral gives Intermediate 29, the cis isomer as a oily compound ¹H NMR (CDCl₃) (delta) (ppm) 7.7 (brs, 1H, NH indole); 7.5 (d, 1H, H aromatic); 7.25-6.9 (m, 3H, H aromatic); 6.15 (d, 1H, H aromatic); 5.85 (m, 1H, H aromatic); 5.25 (brs, 1H, H-1); 4.2 (q, 2H, CO₂CH₂CH₃); 3.8 (dd, 1H, H-3); 3.2-2.8 (m, 2H, H-4); 2.2 (s, 3H, CH₃); 1.25 (t, 3H, CO₂CH₂CH₃) and Intermediate 30, the trans isomer as a cream solid m.p.: 152° C.

### INTERMEDIATES 31 AND 32

### Ethyl 1,2,3,4-Tetrahydro-1-(4-Methylphenyl)-9H-Pyrido (3,4-b)Indole-3-Carboxylate, Cis and Trans Isomers

The same method but starting from racemic tryptophan ethyl ester and p-tolualdehyde gives Intermediate 31, the cis isomer as white crystals m.p.: 148° C. and Intermediate 32, the trans isomer as white crystals m.p.: 180° C.

### INTERMEDIATES 33 AIND 34

### Methyl 1,2,3,4-Tetrahydro-1-(3-Methylphenyl)-9H-Pyrido (3,4-b)Indole-3-Carboxylate, Cis and Trans Isomers

The same method but starting from racemic tryptophan methyl ester and m-tolualdehyde gives Intermediate 33, the cis isomer as white crystals ¹H NMR (CDCl₃) (delta)(ppm): 7.6-7 (m, 9H, H aromatic); 5.2 (brs, 1H, H-1) ; 4-3.9 (dd, 1H, H-3) 3.8 (s, 3H, CO₂CH₃); 3.2-3.1 (ddd, 1H, H-4) 3 (m, 1H, H-4) ; 2.35 (s, 3H, CH₃); 1.7 (brs, 1H, NH) and Intermediate 34, the trans isomer as a white solid m.p.: 175° C.

### INTERMEDIATES 35 AiND 36

### Methyl 1,2,3,4-Tetrahydro-1-(4-Trifluoromethylphenyl)-9H-Pyrido (3,4-b)Indole-3-Carboxylate, Cis and Trans Isomers

The same method but starting from racemic tryptophan methyl ester and 4-trifluoromethylbenzaldehyde gives Intermediate 35, the cis isomer as pale yellow crystals m.p.: 190° C. and Intermediate 36, the trans isomer as pale yellow crystals m.p.: 203° C.

### INTERMEDIATES 37 AiND 38

### Ethyl 1,2,3,4-Tetrahydro-1-(4-Cyanophenyl)-9H-Pyrido (3,4-b)Indole-3-Carboxylate, Cis and Trans Isomers

The same method but starting from racemic tryptophan ethyl ester and 4-cyanobenzaldehyde gives Intermediate 37, the cis isomer as white crystals m.p.: 200° C. and Intermediate 38, the trans isomer as white crystals m.p.: 156° C.

### INTERMEDIATE 39

### Methyl 1,2,3,4-Tetrahydro-1-(4-Hydroxyphenyl)-9H-Pyrido(3,4-b)Indole-3-Carboxylate, Cis Isomer

The same method but starting from racemic tryptophan ethyl ester and 4-hydroxybenzaldehyde gives the title compound as pale yellow crystals ¹H NMR (DMSO) (delta)(ppm): 10.3 (s, 1H, 1NH-indole) 9.4 (s, 1H, OH) ; 7.8-7.5 (m, 8H, H aromatic) ; 5.1 (brs, 1H, H-1) ; 3.9 (m, 1H, H-3) ; 3.75 (s, 3H, CO₂CH₃) 3.1 (m, 1H, H-4) ; 2.8 (m, 1H, H-4).

### INTERiMEDIATE 40

### Methyl 1,2,3,4-Tetrahydro-1-(3-Hydroxy-4-Methoxyphenyl)-9H-Pyrido(3,4-b)Indole-3-Carboxylate, Cis Isomer

The same method but starting from racemic tryptophan methyl ester and 3-hydroxy-4-methoxybenzaldehyde gives the title compound as a yellow solid m.p.: 140-148° C.

### INTERMEDIATE 41

### Methyl 1,2,3,4-Tetrahydro-l-(4-Hydroxy-3-Methoxyphenyl)-9H-Pyrido(3,4-b)Indole-3-Carboxylate, Cis Isomer

The same method but starting from racermic tryptophan methyl ester and 4-hydroxy-3-methoxybenzaldehyde gives the title compound as a cream solid m.p.: 195° C.

### INTERMEDIATE 42

### Methyl 1,2,3,4-Tetrahydro-1-(4-Ethylphenyl)-9H-Pyrido(3,4-b) Indole-3-Carboxylate. Cis and Trans Isomers

The same method but starting from racemic tryptophan methyl ester and 4-ethylbenzaldehyde gives the cis and trans isomer of the title compound. Cis isomer: white solid ¹H NMR (CDCl₃) (delta)(ppm): 7.65-7.1 (m, 9H, H aromatic); 5.25 (brs, 1H, H-1) ; 4(dd, 1H, H-3); 3.9 (s, 3H, CO₂CH₃) ; 3.4 (ddd, 1H, H-4) ; 3.1 (m, 1H, H-4) ; 2.7 (q, 2H, CH₂CH₃) 1.4 (t, 3H, CH₂CH₃). Trans isomer: white solid m.p.: 187° C.

### INTERMEDIATES 43 AND 44

### Methyl 1,2,3,4-Tetrahydro-1-(4-Isopropylphenyl)-9H-Pyrido (3,4-b)Indole-3-Carboxylate. Cis and Trans Isomers

The same method but starting from racemic tryptophan ethyl ester and 4-isopropylbenzaldehyde gives Intermediate 43, the cis isomer as a white solid ¹H NMR (DMSO) (delta)(ppm): 10-15 (s, 1H, NH indole); 7.3-6.7 (m, 8H, H aromatic) ; 5 (brs, 1H, H-1) ; 3.6 (m, 1H, H-3) ; 3.5 (s, 3H, CO₂CH₃) ; 2.95-2.5 (m, 3H, H-4+CH—(Me)₂) 2.4 (brs, 1H, NH) ; 1(d, 6H, 2×CH₃) and Intermediate 44, the trans isomer as a white solid m.p.:189° C.

### INTERIMEDIATES 45 AND 46

### Ethyl 1,2,3,4-Tetrahydro-1-(4-Nitrophenyl)-9H-Pyrido (3,4-b)Indole-3-Carboxylate, Cis and Trans Isomers

The same method but starting from racemic tryptophan ethyl ester and 4-nitrobenzaldehyde gives Intermediate 45, the cis isomer as yellow crystals m.p.: 168° C. and Intermediate 46, the trans isomer as yellow crystals m.p. : 195° C.

### INTERMEDIATE 47

### Ethyl 1,2,3,4-Tetrahydro-l -(4-Dimethylaminophenyl)-9H-Pyrido (3,4-b)Indole-3-Carboxylate Mixture of Cis and Trans Isomers

The same method but starting from racemic tryptophan ethyl ester and 4-dimethylaminobenzaldehyde gives the title compound as white crystals m.p.:170° C.

### INTERMEDIATES 48 AND 49

### Ethyl 1,2,3,4-Tetrahydro-1-(3-Pyridyl)-9H-Pyrido(3,4-b) Indole-3-Carboxylate, Cis and Trans Isomers

The same method but starting from racemic tryptophan ethyl ester and 3-pyridinecarboxaldehyde gives Intermediate 48, the cis isomer as pale yellow crystals m.p.:230-232° C. and Intermediate 49, the trans isomer as white crystals m.p.:210-214° C.

### INTERMEDIATES 50 AND 51

### Methyl 1,2,3,4 Tetrahydro-6-Fluoro-1-(3,4-Methylenedioxyphenyl)-9H-Pyrido(3,4-b) Indole-3-Carboxylate. Cis and Trans Isomers

The same method but starting from racemic 5-fluoro-tryptophan methyl ester and piperonal gives Intermediate 50, the cis isomer as a cream solid m.p.:60° C. and Intermediate 51, the trans isomer as a cream solid m.p.:213° C.

### INTERiMEDLATES 52 AIND 53

### Methyl 1,2,3,4-Tetrahydro-6-Fluoro-1-(4-Methoxyphenyl)-9H-Pyrido(3,4-b)Indole-3-Carboxylate, Cis and Trans Isomers

The same method but starting from racemic 5-fluoro-tryptophan methyl ester and 4-methoxybenzaldehyde gives Intermediate 52, the cis isomer as a solid ¹H NMR (CDCl₃)(delta) (ppm):7.4-6.8 (m, 8H, H aromatic); 5.15 (brs, 1H, H-1) ; 3.9 (dd, 1H, H-3) 3.8 (s,3H, CO₂)CH₃) ; 3.2-2.9 (m, 2H, H-4) and Intermediate 53, the trans isomer as a solid m.p.: 197° C.

### INTERMEDIATES 54 AND 55

### (1R,3R)-Methyl 1,2,3,4-Tetrahydro-1-(3,4-Methylenedioxyphenyl)-9H-Pyrido (3,4-b)Indole-3-Carboxylate, Cis Isomer and (1S,3R)-Methyl 1,2,3,4-Tetrahydro-1-(3,4-Methylenedioxyphenl)-9H-Pyrido(3,4-b)Indole-3-Carboxylate Trans Isomer

To a stirred solution of D-tryptophan methyl ester (11 g) and piperonal (7.9 g) in anhydrous CH₂)Cl₂) (400 ml) cooled at 0° C. is added dropwise trifluoroacetic acid (7.7 ml), and the solution was allowed to react at ambient temperature. After 4 days, the yellow solution is diluted with CH₂Cl₂ (200 ml) and washed with a saturated aqueous solution of NaHCO₃, then with water (3×200 ml) and dried over Na₂SO₄. The organic layer is evaporated under reduced pressure, and the residue is purified by flash chromatography eluting with dichloromethane/ethyl acetate (97/3) to give first Intermediate 54, the cis isomer m.p.:154° C. followed by Intermediate 55, the trans isomer (8.4 g) m.p.:188° C. The following compounds are obtained in a similar manner:

### INTERMEDIATE 56

### (1S, 3S) Methyl- 1,2,3,4-Tetrahydro-1-(3,4-Methylenedioxyphenyl)-9H-Pyrido(3,4-b) Indole-3-Carboxylate, Cis Isomer and (1R, 3S) Methyl-1,2,3,4-Tetrahydro-1-(3,4-Methylenedioxyphenyl)-9H-Pyrido(3,4-b)Indole-3-Carboxylate, Trans Isomer

The same method as for Intermediate 55 but starting from L-tryptophan methyl ester and piperonal gives the cis and trans isomers of the title compound. Cis isomer: white crystals m.p.:154° C. Trans isomer: white crystals m.p.:187-189° C.

### INTERMEDIATES 57 AND 58

### (1R,3R)-Methyl 1,2,3,4-Tetrahydro-1-(4-Methoxyphenyl)-9H-Pyrido(3,4-b) Indole-3-Carboxylate, Cis Isomer and (1S,3R)-Methyl 1,2,3,4-Tetrahydro-1-(4-Methoxyphenyl)-9H-Pyrido(3,4-b)Indole-3-Carboxylate, Trans Isomer

The same method but starting from D-tryptophan methyl ester and 4-methoxybenzaldehyde gives Intermediate 57, the cis isomer as white crystals m.p.: 124-125° C. and Intermediate 58, trans isomer as white crystals m.p.:219-222° C.

### INTERiMEDIATES 59 AND 60

### (1R, 3R)-Methyl 1,2,3,4-Tetrahydro-1-(3-Chloro-4-Methoxyphenyl)-9H-Pyrido(3,4-b)Indole-3-Carboxylate, Cis Isomer and (1S, 3R4-Methoxyphenyl)9H-Pyrido(3,4-b)Indole-3-Carboxylate, Trans Isomer)-Methyl 1,2,3,4-Tetrahydro-1-(3-chloro-

The same method, but starting from D-tryptophan methyl ester and 3-chloro-4-methoxybenzaldehyde gives Intermediate 59, the cis isomer isolated as the hydrochloride salt as white crystals m.p.:200° C. and Intermediate 60, the trans isomer as white crystals m.p.:164° C.

### INTERMEDIATES 61 AND 62

### (1R,3R)-Methyl 1,2,3,4-Tetrahydro-1-(2,3-Dihydrobenzo(b)Furail-5-yl)-9H-Pyrido(3,4-b)Indole-3-Carboxylate, Cis Isomer and(1S,3R)-Methyl 1,2,3,4-Tetrahydro-1-(5-(2,3-Dihydrobenzo(b)Furan))-9H-Pyrido(3,4-b)Indole-3-Carboxylate, Trans Isomer

The same method but starting from D-tryptophan methyl ester and 2,3-dihydrobenzo(b)furan-5-carboxaldehyde gives Intermediate 61, the cis isomer as white crystals m.p.:282° C. and Intermediate 62, the trans isomer as white crystals m.p.:204° C.

### INTERMEDIATES 63 AND 64

### (1R,3R)-Methyl 1,2,3,4-Tetrahydro-1-(5-Indanyl)-9H-Pyrido(3,4-b) Indole-3-Carboxylate Cis Isomer and(1S,3R)-Methyl 1,2,3,4-Tetrahydro-1-(5-Indanyl)-9H-Pyrido(3,4-b)Indole-3-Carboxylate Trans Isomer

The same method but starting from D-tryptophan methyl ester and indan-5-carboxaldehyde gives Intermediate 63, the cis isomer as white crystals m.p.:130-131° C. and Intermediate 64, the trans isomer as white crystals m.p.:196° C.

### INTERIMEDIATE 65

### Ethyl 1,2,3,4-Tetrahydro-1-(4-Trifluoromethoxyphenyl)-9H-Pyrido(3,4-b)Indole-3-Carboxylate, Cis and Trans Isomers

The same method but starting from racemic tryptophan ethyl ester and 4-trifluoromethoxybenzaldehyde gives cis and trans isomers of the title compound. Cis isomer: white crystals m.p.:88° C. Trans isomer: white crystals m.p.:152° C.

### INTERMEDIATE 66

### Methyl 1,2,3,4-Tetrahydro-1-(5-Methyl-2-Thienyl)-9H-Pyrido(3,4-b)Indole-3-Carboxylate, Cis and Trans Isomers

The same method but starting from racemic tryptophan methyl ester and 5-methyl-2-thiophenecarboxaldehyde gives the cis and trans isomers of the title compound. Cis isomer: oily compound ¹H NMR (CDCl₃) (delta) (ppm):8.4 (brs, 1H, NH-indole); 7.7-6.6 (m, 6H, H aromatic); 5.5 (brs, 1H, H-1); 3.9 (dd, 1H, H-3); 3.85 (s, 3H, CO₂CH₃); 3.3-2.9 (m, 2H, H-4); 2.5 (s, 3H, CH₃). Trans isomer: white crystals m.p.:194° C.

### INTERMEDIATES 67 AND 68

### (1S,3R)-Methyl1,2,3,4-Tetrahydro-1-(3,4-Methylenedioxyphenyl)-9H-Pyrido(3,4-b)Indole-3-Carboxylate and (1R, 3R)-Methyl 1,2,3,4-Tetrahydro-1-(3,4-Methylenedioxyphenyl)-9H-Pyrido(3,4-b)Indole-3-Carboxylate

To a stirred solution of D-tryptophan methyl ester (obtained by treating the corresponding hydrochloride salt in water with saturated aqueous NaHCO₃ solution and extraction with CH₂Cl₂ (25.7 g) and piperonal (19.4 g) in anhydrous dichloromethane (700 ml) cooled to 0° C. is added dropwise trifluoroacetic acid (18.1 ml), and the solution is allowed to react at 4° C. After 5 days, the yellow solution is diluted with dichloromethane (500 nm). The organic layer is washed with a saturated aqueous solution of NaHCO₃, then with water (3×500 ml) until the pH is neutral and dried over Na₂SO₄. The organic layer is evaporated under reduced pressure to a volume of about 500 ml. The trans-isomer, which crystallises, is filtered, and the filtrate is reduced to 200 ml. Another fraction of the trans-isomer crystallises. The fractions of trans-isomer are combined to give the (1S,3R) isomer, Intermediate 67, as white crystals. mp: 188° C. ((alpha))20° D=+32.4° (C=1.03, CHCl₃). The filtrate containing mainly the cis-isomer is reduced to 100 ml, and isopropyl ether (200 ml) is added. Upon cooling, the (1R,3R) isomer, Intermediate 68, crystallises as a white solid. mp:154-155° C. ((alpha))20° D=+24.40 (C=1.03, CHCl₃).

### INTERiMEDIATE 69

### (1R,3R)-Methyl 1,2,3,4-Tetrahydro-1-(3,4-Methylenedioxyphenyl)-9H-Pyrido(3,4-b)Indole-3-Carboxylate

Method A

Intermediate 67 (5.0 g) is dissolved in methanol (150 ml). Hydrogen chloride is bubbled into the solution for several minutes at 0° C., and the resulting yellow solution is refluxed for 24 hours. The solvent is removed under reduced pressure and the residue is basified with a saturated aqueous solution of NaHCO₃ and extracted with dichloromethane. The organic layer is washed with water, dried over Na₂SO₄ and purified by flash chromatography eluting with dichloromethane/methanol (99/1) to give the title compound (2.3 g) corresponding to an authentic sample of Intermediate 68.

Method B

Intermediate 67 (25 g) is heated in 1N hydrochloric acid (78.5 ml) and water (400 ml) at 60° C. for 36 hours. From the initial pale yellow solution, a white solid precipitated. The mixture is then allowed to cool to 0° C. and the solid filtered. The solid is then washed with diisopropyl ether (3×200 ml) and dried to give the hydrochloride salt of the title compound as a white solid. m.p. (dec.):209-212° C.

Method C

A 1:1 mixture of the cis and trans isomers of Intermediates 54 and 55 (2 g) is heated in 1N hydrochloric acid (6.8 ml) and water (15 ml) at 50° C. for 72 hours. A similar work-up as described in Method B above gives the hydrochloride salt of the title compound (1.7 g) as a white solid.

### INTERMEDlATE 70

### (R)-N(Alpha))-(3,4-Methylenedioxyphenylcarbonyl)-Tryptophan Methyl Ester

To a suspension of D-tryptophan methyl ester hydrochloride (I0.2 g) in anhydrous CH₂Cl₂ (150 ml) cooled at 0° C. is added dropwise triethylamine (12.3 ml). To the resulting solution solid piperonyloyl chloride (8.16 g) is added portionwise at the same temperature, and the mixture is stirred at room temperature for 2 hours. The mixture is washed successively with water, 0.5N hydrochloric acid, water, a saturated aqueous solution of NaHCO₃ and again with water. After drying over Na₂SO₄ and evaporation of the solvent under reduced presure, the resulting oil on trituration from hot cyclohexane afforded the title compound as a white solid (14.7 g). m.p.:123-124° C. ((alpha))20° D=−844° (c=104, CHCl₃)

### INTERMEDIATE 71

### (R)-N(Alpha))-(3,4-Methylenedioxyphenylthiocarbonal)-Tryptophan Methyl Ester

A mixture of Intermediate 70 (14 g) and Lawesson's reagent (9.28 g) in dimethoxyethane (280 ml) is heated at 60° C. under N₂ for 16 hours with stirring. The reaction mixture is evaporated to dryness and the resulting oil is dissolved in ethyl acetate, then washed successively with an aqueous saturated solution of NaHCO₃ and water and dried over Na₂SO₄. The oily residue obtained after evaporation under reduced pressure gives, on trituration from cyclohexane, a yellow powder which is filtered and washed with cooled methanol to afford the title compound. mp: 129-130° C. ((alpha))20° D=−186.8° (c=1.14, CHCl₃).

### INTERiMEDIATE 72

### (1R,3R)-Methyl 1,2,3,4-Tetrahydro-1-(3,4-Methylenedioxyphenyl)-9H-Pyrido(3,4-b)Indole-3-Carboxylate

A solution of Intermediate 71 (9 g) and methyl iodide (10 ml) in anhydrous dichloromethane (200 ml) is heated at reflux under an argon atmosphere with protection from light. After 24 hours, the solvent is removed under reduced pressure to give an orange oil which on trituration from hexane gives a solid which is washed with ether and used without further purification in the next step. This compound is dissolved in methanol (250 ml) and the solution is cooled to −78° C. NaBH₄ (0.99 g) is then added by portions, and the mixture is stirred at the same temperature for 1 hour. The reaction is quenched by addition of acetone (10 ml), and the solvent is removed under reduced pressure. The residue is dissolved in CH₂Cl₂, washed with water and then with brine and dried over Na₂SO₄. After evaporation of the solvent, the orange oil gives on trituration from a hot mixture of diethyl ether/cyclohexane an orange powder which is recrystallised from diethyl ether/pentane to afford the title compound as a pale yellow solid corresponding to a sample of Intermediate 68.

### INTERMEDIATE 73

### (1R,3R)-Methyl 1,2,3,4-Tetrahydro-2-Chloroacetyl-(3,4-Methylenedioxyphenyl)-9H-Pyrido(3,4-b)Indole-3-Carboxylate

Method A

To a stirred solution of Intermediate 72 (9.7 g) and NaHCO₃ (2.79 g) in anhydrous CHCl₃ (200 ml) is added dropwise chloroacetyl chloride (5.3 ml) at 0° C. under N₂. The resulting mixture is stirred for 1 hour at the same temperature and diluted with CHCl₃ ( 100 ml). Water (100 ml) is then added dropwise with stirning to the mixture, followed by a saturated aqueous solution of NaHCO₃. The organic layer is washed with water until neutrality and dried over Na₂SO₄. After evaporation of the solvent under reduced pressure, the oily compound obtained is crystallised from ether to give the title compound as a pale yellow solid. mp : 233° C. ((alpha))20° D=−125.4° (c=1.17, CHCl₃).

Method B

Chloroacetyl chloride (4 ml) is added dropwide to a solution of Interrnediate 72 (16.1 g) and triethylamine (7 ml) inmanhydrous CH₂Cl₂ (200 ml) at 0° C. under N₂. The solution is stirred at 0° C. for 30 minutes, then diluted with CH₂Cl₂ (300 m1). The solution is washed with water (200 ml), a saturated aqueous solution of NaHCO₃ (300 ml) and brine (400 ml). After drying over Na₂SO₄ and evaporation under reduced pressure, the resulting solid is washed with ether (300 ml) to give the title compound as a pale yellow solid.

### INTERMEDIATE 74

### Methyl 1,2,3,4-Tetrahydro-6-Methyl-1-(3,4-Methylenedioxyphenyl)-9H-Pyrido(3,4-b)Indole-3-Carboxylate, Cis and Trans Isomers

The cis and trans isomers of the title compound are prepared using the method described in Intermediate 1 but starting from racemic 5-methyltryptophan methyl ester and piperonal. Cis isomer: yellow solid m.p.: 85° C. Trans isomer: yellow solid m.p.: 185° C.

### INTERMEDIATES 75 AND 76

### (1R, 3R)-Methyl 1,2,3,4-Tetrahydro-1-(7-(4-Methyl-3,4-Dihydro-2H-Benzo(1,4)Oxazinyl))-9H-Pyrido(3,4-b)Indole-3-Carboxylate, Cis Isomer and (1S, 3R)-Methyl1,2,3,4-Tetrahydro-1-(7-(4-Methyl-3,4-Dihydro-2H-Benzo(1,4)Oxazinyl))-9H-Pyrido(3,4-b)Indole-3-Carboxylate, Trans Isomer

The same method, as described for intermediates 54 and 55, but starting from D-tryptophan methyl ester and 4-methyl-3,4-dihydro-2H-benzo(1,4)oxazine-7-carboxaldehyde gives Intermediate 75 the cis isomer as an oily compound ¹H NMR (CDCl₃) (delta) (ppm) : 7.6-7.1 (m, 5H) ; 6.9-6.6 (m, 3H) ; 5.15 (br s, 1H) ; 4.3 (t, 2H) ; 4 (dd, 1H) ; 3.8 (s, 3H); 3.3 (t, 2H) ; 3.3-2.95 (m, 2H) ; 2.9 (s, 3H); 1.6 (br s) and intermediate 76, the trans isomer as white crystals m.p.: 119-121° C.

### INTERMEDIATE 77

### Methyl 1,2,3,4-Tetrahydro-1-(5-(N-Benzylindolinyl))-9H-Pyrido(3,4-b)Indole-3-Carboxylate, Mixture of (1R, 3R) and (1S, 3R) Isomers

The same method, as described for Intermediates 54 and 55, but starting from D-tryptophan methyl ester and N-benzylindoline-5-carboxaldehyde gives intermediate 77 as an oily compound.

### INTERMEDIATES 78 AND 79

### (1R, 3R)-Methyl 1,2,3,4-Tetrahydro-1-(4-Carbomethoxyphenyl)-9H-Pyrido (3,4-b)Indole-3-Carboxylate, Cis Isomer and(1S, 3R)-Methyl 1,2,3,4-Tetrahydro- 1-(4-Carbomethoxyphenyl)-9H-Pyrido(3,4-b)Indole-3-Carboxylate, Trans Isomer

The same method, as described for Intermediates 54 and 55, but starting from D-tryptophanmethyl ester and methyl 4-formylbenzoate gives Intermediate 78, the cis isomer as white crystals m.p. 157-160° C. and Intermediate 79, the trans isomer as pale yellow crystals m.p.: 124-126° C.

### INTERMEDIATE 80

### (1R, 3R)-Methyl 1,2,3 ,4-Tetrahydro-2-(2-(Benzyloxycarbonyl)-R-Prolyl)-1-(3,4-Methylenedioxyphenyl)-9H-Pyrido(3,4-b)Indole-3-Carboxylate

A solution of N-(benzyloxycarbonyl)-D-proline acid chloride (0.64 g, 2.4 mmol) in anhydrous dichloromethane (10 ml) is added dropwise to a stirred solution of intermediate 54 (0.7 g, 2 mmol) and triethylamine (0.33 ml, 2.4 mmol) in dichloromethane (15 ml) at −10° C. The mixture is stirred for 2 hours at −10° C. after which it is diluted with dichloromethane (50 ml), washed with hydrochloric acid (1N), water, a saturated solution of NaHCO₃, a saturated NaCI solution and dried over Na₂SO₄. Evaporation of the solvent and recrystallisation of the crude product from methanol gives the title compound as pale yellow crystals (0.75 g) m.p.: 268-270° C.

### INTERiMEDIATE 81

### (1R, 3R)-Methyl 1,2,3,4Tetrahydro-2-(2-(Benzyloxycarbonyl)-S-Prolyl)-1-(3,4-Methylenedioxyphenyl)-9H-Pyrido(3,4-b)Indole-3-Carboxylate

A solution of N-(enzyloxycarbonyl)-L-proline acid chloride (0.86 g, 3.2 mmol) in anhydrous dichloromethane (10 ml) is added dropwise to a stirred solution of intermediate 54 (0.91 g, 2.6 mmol) and triethylamine (0.44 ml, 3.2 mmol) in dichloromethane (20 ml) at −10° C. The mixture is stirred for 2 hours at −10° C. after which it is diluted with dichloromethane (60 ml), washed with hydrochloric acid (1N), water, a saturated solution of NaHCO₃, a saturated NaCl solution and dried over Na₂SO₄. Evaporation of the solvent and recrystallisation of the crude product from methanol/water gives the title compound as pale yellow crystals m.p.: 115-120° C.

### INTERiMEDIATE 82

### (1R, 3R)-Methyl 1,2,3,4-Tetrahydro-2-(2-Chloropropionyl)-1-(3,4-Methylenedioxyohenyl)-9H-Pyrido(3,4-b)Indole-3-Carboxylate

To a solution of (S)-(−)-2-chloropropionic acid (87 μl, 1 mmol) in anhydrous dichloromethane (15 ml), is added dicyclohexylcarbodiimide (0.23 g, 1.1 mmol). Intermediate 54 (0,35 g, 1 mmol) is then added and the mixture isstirred at room temperature for 20 hours. The formed precipitate of dicyclohexylurea is removed by filtration, the filtrate is evaporated in vacuo and the crude product is purified by flash chromatography eluting with toluene/ethyl acetate: 95/5. The oily compound obtained is then crystallised from ether/hexane to give the title compound as pale yellow crystals (0.31 g) m.p.: 125-127° C.

### INTERMEDIATE 83

### (1R, 3R)-Methyl 1,2,3,4-Tetrahydro-2-(2-Chloropropionyl)-1-(3,4-Methylenedioxyohenyl)-9H-Pyrido(3,4-b)Indole-3-Carboxylate

To a solution of (R)-(+)-2-chloropropionic acid (191 μl, 2.2 mmol) in anhydrous dichloromethane (30 ml), is added dicyclohexylcarbodiimide (0.45 g, 2.2. mol). Intermediate 54 (0,7 g, 2 mmol) is then added and the mixture is stirred at room temperature for 20 hours. The formed precipitate of dicyclohexylurea is removed by filtration, the filtrate is evaporated in vacuo and the crude product is purified by flash chromatography eluting with toluene/ethyl acetate: 95/5. The oily compound obtained is then crystallised from ether/hexane to give the title compound as pale yellow crystals (0.74 g) m.p.: 126-128° C.

### INTERMEDLATES 84 AND 85

### (1R, 3R)-Methyl 1,2,3,4-Tetrahydro- 1-(3,4-Dibenzyloxyphenyl)-9H-Pyrido (3,4-b)Indole-3-Carboxylate Cis Isomer and (1S, 3R)-Methyl 1,2,3,4-Tetrahydro-1-(3,4-Dibenzyloxyohenyl)-9H-Pyrido (3,4-b)Indole-3-Carboxylate Trans Isomer

The same method as described for intermediates 54 and 55 but starting from D-tryptophan methyl ester and 3,4-dibenzyloxybenzaldehyde gives intermediate 84, the cis isomer as an oily compound ¹H NMR (CDCl₃) (delta)(ppm):7.5-6.95 (m, 15H) ; 6.85 (s, 1H) ; 6.75 (s, 2H) ; 5.1 (s, 2H) ; 5 (br s, 1H) ; 4.95 (d, 2H) 3.85 (dd, 1H) ; 3.7 (s, 3H); 3.2-2.8 (m, 2H) ; 2.3 (br s, 1H) and intermediate 85, the trans isomer as an oily compound ¹HNMR (CDCl₃)(delta) (ppm) 7.6-7 (m, 15H); 6.9-6.7 (m,3H);5.2(brs, 1H);5.1 (s,2H);5(s,2H);3.8(t, 1H);3.65 (s,3H);3.3-3(m, 2H); 2.25 (br s, 1H).

### INTERMEDIATE 86

### (6R,12aR)-2,3,6,7,12,12a-Hexahydro-6-(3,4-Dibenzyloxyphenyl)-2-Methylpyrazino(2′,1′:6,1)Pyrido(3,4-b)Inle-1,4-Dione

The same two-step procedure but starting from intermediate 84 and methylamine gives, after recrystallisation from dichloromethane/ether, the title compound as white crystals m.p.:158-160° C., ((alpha))20°)D))=+11.7°(c=1.23 CHCl₃).

### INTERMEDIATE 87

### Methyl 1,2,3,4-Tetrahydro-1-(5-(2-Methylisoindolinyl))-9H-Pyrido (3,4-b)Indole-3-Carboxylate, Mixture of (1R,3R) and (1S,3R) Isomers

The same method, as described for intermediates 54 and 55, but starting from D-tryptophan methyl ester and N-methylisoindoline-5-carboxaldehyde gives intermediate 87 as an oily compound.

### INTERMEDIATES 88 AND 89

### (1R, 3R)-Methyl 1, 2, 3, 4-Tetrahydro-1-(5-Benzofuranyl)-9H-Pyrido[3,4-b]Indole-3-Carboxylate, Cis Isomer; and (1S, 3R)-Methyl-1, 2, 3, 4-Tetrahydro-1-(5-Benzofuranyl)-9H-Pyrido[3,4-b]Indole-3-Carboxylate Trans Isomer

To a stirred solution of D-tryptophan methyl ester (3.73 g) and 5-formylbenzofuran′ (2.5 g)(prepared as is described in Chimie Therapeutique 4, pp 221-227 (1966)) in anhydrous dichloromethane (100 ml) cooled at 0° C. was added dropwise trifluoroacetic acid (2.63 ml), and the solution was allowed to react at ambient temperature. After 72 hours, the solution was washed with a saturated aqueous solution of NaHCO₃, then with water and dried over Na₂SO₄. The organic layer was evaporated under reduced pressure, and the residue was purified by flash chromatography efuting with dichloromethane/ethyl acetate (90/10) to give first the cis isomer (Intermediate 1) (3 g) as an amorphous compound, followed by the trans isomer (intermediate 2) (2.5 g) as white crystals, m.p. 194-195° C.

### INTERMEDIATE 90

### (1R, 3R)-Methyl 1, 2, 3, 4-Tetrahydro-l-(5-Benzofuranyl)-2-Chloroacetvl-9H-Pyrido [3,4-b]Indole-3-Carboxylate

To a stirred solution of Intermediate 1 (2 g) and triethylamine (0.88 ml) in anhydrous dichloromethane (40 mL) cooled at 0° C. was added dropwise chloroacetylchloride (0.5 ml), and the solution was stirred at the same temperature for 1 hour. The solution was washed with water, dried over Na₂SO₄ and evaporated to dryness, and the residue was crystallized from methanol to give the title compound (1.8 g) as pale yellow crystals. m.p.: 227-228° C.

### INTERMEDIATE 91

### (1 R, 3R)-Methyl 1, 2, 3, 4-Tetrahydro-l-(5-Denzofuranyl)-2-(2-(S)-Benzyloxycarbonylaminopropionyl)-9H-Pyrido[3,4-b]Indole-3-Carboxylate

To a stirred solution of (S)-2-benzyloxycarbonylaminopropionic acid (1.3 g) and 1,3-dicyclohexyl carbodiimide (DCC) (1.2 g) in anhydrous dichloromethane (50 ml) at 0° C. was added Intermediate 1 (1.0 g). The resulting mixture was stirred for 72 hours then the resulting precipitate filtered off. The filtrate was evaporated to dryness and the residue purified by flash chromatography, eluting with cyclohexane/ethyl acetate (60/40) to give the title compound as white crystals (1.4 g) m.p. : 91-92° C.

### INTERMEDIATE 92

### (1R, 3R)-Methyl 1, 2, 3, 4-Tetrahydro-1-(5-Benzofiuranyl)-2-[2-(S)-Benzyloxycarbonylmethylamino)Propionyl]-9H-Pyrido[3,4-b]Indole-3-Carboxylate

The same procedure employed in the preparation of Intermediate 4 but starting from 2-(S)-benzyloxycarbonylmethylamino)propionic acid (0.82 g) and using Intermediate 1 (0.6 g), DCC (0.72 g) and dichloramethane (25 ml) gives after chromatography, eluting with cyclohexane/ethyl acetate (70/30), the title compound as a white foam.

### EXAMPLE 1

### Cis-2,3,6,7,12,12a-Hexahydro-2-Methyl-6-(3,4-Methylenedioxyphenyl) Pyrazino(2′,1′:6,1)Pyrido(3,4-b)Indole-1,4-Dione

(a) To a stirred solution of intermediate 1 (2 g) and NaHCO₃ (0.6 g) in anhydrous CHCl₃ (40 ml) is added dropwise chloroacetyl chloride (1.1 ml) at 0° C. The resulting mixture is stirred for 1 hour at the same temperature and diluted with CHCl₃. Water (20 ml) is then added dropwise with stirring to the mixture, followed by a saturated solution of NaHCO₃. The organic layer is washed with water until neutrality and dried over Na₂SO₄. After evaporation of the solvent under reduced pressure, cis-methvl 1.2.3.4 tetrahydro-2-chloroacetyl-l-(3,4-methylenedioxyphenyl)-9H-pyrido(3,4-b) indole-3-carboxylate is obtained as an oil which is crystallised from ether (2 g, m.p.: 215-218° C.) and is used without further purification in the next step.

(b) To a stirred suspension of the chloroacetyl intermediate (0.34 g) in MeOH (20 ml) is added at ambient temperature a solution of methylamine (33% in EtOH) (0.37 ml) and the resulting mixture is heated at 50° C. under N₂ for 14 hours. The solvent is removed under reduced pressure and the residue is dissolved in CH₂Cl₂ (50 ml). After washing with water (3×30 ml), drying over Na₂SO₄ and evaporating to dryness, the residue is purified by flash chromatography eluting with CH₂Cl₂/MeOH (99/1) and recrystallised from MeOH to give the title compound as white crystals (0.19 g) m.p. : 253-255° C. Analysis for C₂₂H₁₉N₃O₄: Calculated:C,67.86;H,4.92;N,10.79; Found:C,67.53 ;H,4.99;N,10.62%.

The following compounds are obtained in a similar manner:

### EXAMPLE 2

### Cis-2,3,6,7,12,12a-Hexahydro-2-Butyl-10-Fluoro-6-(4-Methoxyphenyl)Pyrazino(2′,1′:6,1 )Pyrido (3.4-b)Indole-1,4-Dione

The same two-step procedure but starting from butylatnine and intermediate 52 gives, after recrystallisation from ethanol, the title compound as white crystals m.p.: 182° C. Analysis for C₂₅H₂₆FN₃O₃ (0.1 H₂O): Calculated: C, 68.67; H, 6.04; N, 9.61; Found: C, 68.38; H, 6.11; N, 9.53%.

### EXAMPLE 3

### Trans-2,3,6,7,12,12a-Hexahydro-2-Methyl-6-(3,4-Methylenedioxyphenyl)Pyrazino(2′,1′:6,1)Pyrido(3,4-b)Indole-1,4-Dione

The same two-step procedure but starting from methylamnine and intermediate 2 gives, after recrystallisation from toluene, the title compound as white crystals m.p.:301-303° C. Analysis for C₂₂H₁₉N₃O₄: Calculated: C,67.86;H,4.92;N,10.79; Found:C,67.98;H,4.98;N,10.73%.

### EXAMPLE 4

### Cis-2,3,6,7,12,12a-Hexahydro-6-(3,4-Methylenedioxyphenyl) Pyrazino(2′,1′:6,1)Pyrido(3,4-b)Indole-1,4-Dione

The same two-step procedure but starting from ammonia and intermediate 1 gives, after recrystallisation from methanol, the title compound as white crystals m.p.:283-285° C. Analysis for C₂₁H₁₇N₃O₄: Calculated: C,67.19;H,4.56;N,11.19; Found:C,67.04;H,4.49;N,11.10%.

### EXAMPLE 5

### Cis-2,3,6,7,12,12a-Hexahydro-10-Fluoro-6-(4-Methoxyphenyl)-2-(2,2,2-Trifluoroethyl)-Pyrazino(2′,1′:6,1)Pyrido(3,4-b)Indole-1,4-Dione

The same two-step procedure but starting from 2,2,2-trifluoroethylamine and intermediate 52 gives, after recrystallisation from ethanol/diisopropyl ether, the title compound as white crystals m.p.: 190° C. Analysis for C₂₃H₁₉F₄N₃O₃: Calculated: C, 59.87; H, 4.15; N, 9.11; Found: C, 59.81; H, 4.18; N, 9.21%.

### EXAMPLE 6

### Cis-2,3,6,7,12,12a-Hexahydro-10-Fluoro-2-Methyl-6-(3,4-Methylenedioxyphenyl-Pyrazino(2′,1′:6,1)Pyrido(3,4-b)Indole-1,4-Dione

The same two-step procedure but starting from methylamine and intermediate 50 gives, after recrystallisation from ethanol, the title compound as white crystals m.p.: 292° C. Analysis for C2₂H₁₈FN₃O₄: Calculated: C, 64.86 ; H, 4.45 ; N, 10.31; Found: C, 64.66 ; H, 4.60 ; N, 10.21%.

### EXAMPLE 7

### (6R, 12aS)-2,3,6,7,12,12a-Hexahydro-2-Methyl-6-(3,4-Methylenedioxyphenyl)-Pyrazino(2′,1′:6,1)Pyrido(3,4-b)Indole-1,4-Dione

The same two-step procedure but starting from methylamine and the trans isomer of intermediate 56 gives, after recrystallisation from toluene, the title compound as white crystals m.p. :287-289° C. Analysis for C2₂H₁₉N₃O₄ (0.25 toluene): Calculated: C, 69.16; H, 5.13; N, 10.19; Found: C,69.09; H, 5.14; N, 10.19%. ((alpha))20° D=−293.4°(C=1.28, CHCl₃).

### EXAMPLE 8

### (6S, 12aR)-2,3,6,7,12,12a-Hexahydro-2-Methyl-6-(3,4-Methylenedioxyphenyl)Pyrazino(2′,1′:6,1)Pyrido(3,4-b)Indole-1,4-Dione

The same two-step procedure but starting from methylamine and intermediate 55 gives, after recrystallisation from toluene, the title compound as white crystals m.p.:287° C. Analysis for C₂₂H₁₉N₃O₄ (0.3 toluene): Calculated: C, 69.41; H, 5.17 ; N, 10.08; Found: C, 69.56; H,5.24; N, 10.08%. ((alpha))20°D=+297.9° (C=1.21; CHCl₃).

### EXAMPLE 9

### Cis-2,3,6,7,12,12a-Hexahydro-2-(2-(2-Pyridyl)-Ethyl)6-(3,4-Methylenedioxyphenyl)-Pyrazino(2′,1′-6,1)Pyrido(3,4-b)Indole-1,4-Dione

The same two-step procedure but starting from 2-(2-pyridyl)ethylamine and intermediate 1 gives, after recrystallisation from 2-propanol, the title compound as white crystals m.p. :218-222° C. Analysis for C₂₈H2₄N₄O₄; Calculated: C, 69.99 ; H, 5.03 ; N, 11.66; Found: C, 69.92 ; H, 5.16 ; N, 11.48%.

### EXAMPLE 10

### Cis-2,3,6,7,12,12a-Hexahydro-2-(2-Pyridylmethyl)-6-(3,4-Methylenedioxyphenyl-Pyrazino(2′,1′:6,1)Pyrido(3,4-b)Indole-1,4-Dione

The same two-step procedure but starting from 2-pyridyltmethylamine and intermediate 1 gives, after recrystallisation-from DMF/water, the title compound as cream crystals m.p : 285-286° C. Analysis for C₂₇H₂₂N₄O₄ (0.4 H₂O): Calculated: C, 68.46; H,4.85; N, 11.83; Found: C, 68.58; H, 4.88; N, 11.90%.

### EXAMPLE 11

### Cis-2,3,6,7,12,12a-Hexahydro-2-(3-Pyridylmethyl)-6-(3,4-Methylenedioxyphenyl)-Pyrazino(2′,1′:6,1 )Pyrido(3,4-b)Indole-1,4-Dione

The same two-step procedure but starting from 3-pyridylmethylamine and intermediate 1 gives, after recrystallisation from CH₂Cl₂/MeOH, the title compound as cream crystals m.p.: 292-293° C. Analysis: C₂₇H₂₂N₄O₄: Calculated: C, 69.52 ; H, 4.75 ; N, 12.01; Found: C, 69.27 ; H, 4.74 ; N, 11.37%.

### EXAMPLE 12

### Cis-2,3,6,7,12,12a-Hexahydro-2-(4-Pyridylmethyl)-6-(3,4-Methylenedioxyphenyl)-Pyrazino(2′,1′:6,1)Pyrido(3,4-b)Indole-1.4-Dione

The same two-step procedure but starting from 4-pyridylmethylamine and intermediate 1 gives, after recrystallisation from MeOH, the title compound as pale yellow crystals m.p.: 273-274° C. Analysis for C₂₇H₂₂N₄O₄ (1.8 H₂O): Calculated: C, 65.00; H, 5.17; N, 11.23; Found: C, 65.11; H, 4.85; N, 11.07%.

### EXAMPLE 13

### Cis-2,3,6,7,12,12a-Hexahydro-2-Ethyl-6-(3,4-Methylenedioxyphenyl) Pyrazino(2′,1′:6,1Pyrido(3,4-b)Indole-1,4-Dione

The same two-step procedure but starting from ethylamine and intermediate 1 gives, after recrystallisation from methanol, the title compound as white crystals m.p.:272-274° C. Analysis for C₂₃H₂₁)N₃O₄. Calculated: C,68.47;H,5.25;N, 10.42; Found:C,68.52;H,5.35;N,10.53%.

### EXAMPLE 14

### Cis-2,3,6,7,12,12a-Hexahydro-2-(2,2,2-Trifluoroethyl)-6-(3,4-Methylenedioxyphenyl)-Pyrazino(2′,1′:6,1)Pyrido(3,4-b)Indole-1,4-Dione

The same two-step procedure but starting from 2,2,2-trifluoroethylamine and intermediate 1 gives, after recrystallisation from EtOH, the title compound as white crystals m.p.: 303° C. Analysis for C₂₃H₁₈F₃N₃O₄: Calculated: C,60.40;H,3.97;N,9.19; Found:C,60.43;H,4.15;N,9.16%.

### EXAMPLE 15

### Cis-2,3,6,7,12,12a-Hexahydro-6-(3,4-Methylenedioxyphenyl)-2-Propylpyrazino(2′,1′:6,1)Pyrido(3,4-b)Indole-1,4-Dione

The same two-step procedure but starting from propylamine and intermediate 1 gives, after recrystallisation from methanol, the title compound as white crystals m.p. : 270-271° C. Analysis for C₂₄H₂₃N₃O₄: Calculated: C,69.05;H,5.55;N,10.07; Found:C,69.22;H,5.50;N,9.80%.

### EXAMPLE 16

### Cis-2,3,6,7,12,12a-Hexahydro-2-Isopropyl-6-(3,4-Methylenedioxyphenyl) Pyrazino(2′,1′:6,1)Pyrido(3,4-b)Indole-1,4-Dione

The same two-step procedure but starting from isopropylamine and intermediate 1 gives, after recrystallisation from methanol, the title compound as white crystals m.p.: 248-250° C. Analysis for C₂₄H₂₃N₃O₄: Calculated: C,69.05;H,5.55;N,10.07; Found:C,68.86;H,5.66;N,10.21%.

### EXAMPLE 17

### Cis-2,3,6,7,12,12a-Hexahydro-2-Cyclopropyl-6-(3,4-Methylenedioxyphenyl) Pyrazino(2′,1′:6,1)Pyrido(3,4-b)Indole-1,4-Dione

The same two-step procedure but starting from cyclopropylamine and intermediate 1 gives, after recrystallisation from methanol, the title compound as white crystals m.p.: 290-292° C. Analysis for C2_{4H21})N₃O₄: Calculated: C,69.39;H,5.10;N,10.11; Found:C,69. 11 ;H,5.20;N,9.94%.

### EXAMPLE 18

### Cis-2,3,6,7,12,12a-Hexahydro-2-Butyl-6-(3,4-Methylenedioxyphenyl) Pyrazino(2′,1′:6,1)Pyrido(3,4-b)Indole-1,4-Dione

The same two-step procedure but starting from butylamniie and intermediate 1 gives, after recrystallisation from methanol/water, the title compound as white crystals m.p.: 241-243° C. Analysis for C₂₅₁H₂₅₁N₃O₄: Calculated: C,69.59;H,5.84;N,9.74; Found:C,69.77;H,5.82;N,9.81 %.

### EXAMPLE 19

### Trans-2,3,6,7,12,12a-Hexahydro-2-Butyl-6-(3,4-Methylenedioxyphenyl) Pvrazino(2′,1′:6,1)Pyrido(3,4-b)Indole-1,4-Dione

The same two-step procedure but starting from butylamine and intermediate 2 gives, after recrystallisation from toluene, the title compound as white crystals m.p.: 243° C. Analysis for C₂₅H₂₅N₃O₄: Calculated: C,69.59;H,5.84;N,9.74; Found:C,69.80;H,5.78;N,9.52%.

### EXAMPLE 20

### Cis-2,3,6,7,12,12a-Hexahydro-2-Cyclopropylmethyl-6-(3,4-Methylenedioxyphenyl)-Pyrazino(2′,1′:6,1)Pyrido(3,4-b)Indole-1,4-Dione

The same two-step procedure but starting from cyclopropylmethylamine and intermediate 1 gives, after recrystallisation from methanol, the title compound as white crystals m.p.: 217-218° C. Analysis for C₂₅H2₃N₃O₄: Calculated: C,69.92;H,5 .40;N,9.78; Found:C,70.02;H,5.47;N,9. 84%.

### EXAMPLE 21

### Cis-2,3,6,7,12,12a-Hexahydro-2-Cyclopentyl-6-(3,4-Methylenedioxyphenyl)-Pyrazino(2′,1′:6,1)Pyrido(3,4-b)Indole-1,4-Dione

The same two-step procedure but starting from cyclopentylamine and intermediate 1 gives, after recrystallisation from acetone, the title compound as white crystals m.p.: 270° C. Analysis for C₂₆H₂₅₁N₃O₄: Calculated: C,70.41;H,5.68;N,9.47; Found:C,70.58; H,5.63; N,9.38%.

### EXAMPLE 22

### Cis-2,3,6,7,12,12a-Hexahydro-2-Cyclohexyl-6-(3,4-Methylenedioxyphenyl)Pyrazino(2′,1′:6,1)Pyrido(3,4-b)Indole-1,4-Dione

The same two-step procedure but starting from cyclohexylamine and intermediate 1 gives, after recrystallisation from methanol/water, the title compound as white crystals m.p.: 68-269° C. Analysis for C₂₇H₂₇N₃O₄: Calculated: C,70.88;H,5.95;N,9. 18; Found:C,70.82;H,5.89;N,⁹.²¹%.

### EXAMPLE 23

### Cis-2,3,6,7,12,12a-Hexahydro-2-Benzyl-6-(3,4-Methylenedioxyphenyl)Pyrazino(2′,1′:6,1)Pyrido(3,4-b)Indole-1,4-Dione

The same two-step procedure but starting from benzylamine and intermediate 1 gives, after recrystallisation from dichloromethane/hexane, the title compound as white crystals m.p. :285-287° C. Analysis for C₂₈H2₃N₃O₄(1 H₂O): Calculated: C,69.55;H,5.21;N,8.69; Found:C,69.30;H,5.06;N,8.⁴8%.

### EXAMPLE 24

### Cis-2,3,6,7,12,12a-Hexahydro-2-(4-Fluorobenzyl)-6-(3,4-Methylenedioxyphenyl)Pyrazino(2′,1′:6,1)Pyrido(3,4b)Indole-1,4-Dione

The same two-step procedure but starting from 4-fluorobenzylamine and intermediate 1 gives, after recrystallisation from acetone, the title compound as white crystals m.p.: 281-283° C. Analysis for C₂8H₂₂FN₃O₄: Calculated: C,69.56;H,4.59;F,3.93;N,8.69; Found:C69.54;H,4.58; F,3.82;N,8.63%.

### EXAMPLE 25

### Cis-2,3,6,7,12,12a-Hexahydro-6-(4-Methoxyphenyl)-2-Methylpvrazino(2′,1′:6,1)Pyrido(3,4b)Indole-1,4-Dione

The same two-step procedure but starting from methylamine and intermediate 3 gives, after recrystallisation from 2-propanol, the title compound as white crystals m.p.: 257-263° C. Analysis for C₂₂H₂₁N₃O₃: Calculated: C,70.38;H,5.64;N,11.19; Found:C,70.11;H,5.55;N,11.15%.

### EXAMPLE 26

### Trans-2,3,6,7,12,12a-Hexahydro-6-(4-Methoxyphenyl)-2-Methylpyrazino(2′,1′:6,1)Pyrido(3,4-b)Indole- 1,4-Dione

The same two-step procedure but starting from methylamine and intermediate 4 gives, after recrystallisation from diisopropyl ether, the title compound as white crystals m.p.:225-228° C. Analysis for C₂₂H₂₁N₃O₃: Calculated: C,70.38;H,5.64;N,11:19; Found: C,70.34;H,5.77;N,11.19%.

### EXAMPLE 27

### Cis-2,3,6,7, 12,1 2a-Hexahydro-2-Ethyl-6-(4-Methoxyphenyl)Pyrazino (2′,1′:6,1)Pyrido(3,4-b)Indole-1,4-Dione

The same two-step procedure but starting from ethylamine and intermediate 3 gives, after recrystallisation from methanol, the title compound as white crystals m.p.:245-255° C. Analysis for C₂₃H₂₃N₃O₃: Calculated: C,70.93;H,5.95;N,10.79; Found:C,70.74;H,6.06;N,10.87%.

### EXAMPLE 28

### Cis-2,3,6,7,12,12a-Hexahydro-6-(4-Methoxyphenyl)-2-(2,2,2-Trifluoroethyl)Pyrazino(2′,1′:6,1)Pyrido(3,4-b)Indole-1,4-Dione

The same two-step procedure but starting from 2,2,2-trifluoroethylamine and intermediate 3 gives, after recrystallisation from ethanol, the title compound as white crystals m.p.: 232° C. Analysis for C₂₃H₂₀F₃N₃O₃: Calculated: C,62.30;H,4.55 ;N,9.48; Found:C,62.08;H,4.66;N,9.54%.

### EXAMPLE 29

### Cis-2,3,6,7,12,12a-Hexahydro-2-Butyl-6-(4-Methoxyphenyl) Pyrazino(2′,1′:6,1)Pyrido(3,4-b)Indole-1,4-Dione

The same two-step procedure but starting from butylarine and intermediate 3 gives, after recrystallisation from methanol,the title compound as white crystals m.p.:157° C. Analysis for C₂₅H₂₇N₃O₃(0.5 H₂O): Calculated: C,70.40;H,6.62;N,9.85; Found:C,70.25;H,6.60;N,9.83%.

### EXAMPLE 30

### Trans-2,3,6,7,12,12a-Hexahydro-2-Butyl-6-(4-Methoxyphenyl) Pyrazino(2′,1′:6,1l)Pyrido(3,4-b)Indole-1,4-Dione

The same two-step procedure but starting from butylarnine and intermediate 4 gives, after recrystallisation from methanol, the title compound as white crystals m.p.:212-214° C. Analysis for C₂₅H₂₇N₃O₃: Calculated: C,71.92;H,6.52;N,10.06; Found:C,71.81;H,6.55;N,10.03%.

### EXAMPLE 31

### Cis-2,3,6,7,12,12a-Hexahydro-6-(4-Methoxyphenyl)-2-Cyclopropylnethylpyrazino(2′,1′:6,1l)Pyrido(3,4-b)Indole-1,4-Dione

The same two-step procedure but starting from cyclopropylmethylamine and intermediate 3 gives, after recrystallisation from methanol, the title compound as white crystals m.p.: 180-185° C. Analysis for C₂₅H₂₅N₃O₃ (0.5H₂O): Calculated: C,70.74;H,6.17;N,9.90; Found:C, 70.91; H, 6.16; N, 9.80%.

### EXAMPLE 32

### Cis-2,3,6,7,12,12a-Hexahydro-2-Benzyl-6-(4-Methoxyphenyl)Pyrazino(2′,1′:6,1)Pyrido(3,4-b)Indole-1,4-Dione

The same two-step procedure but starting from benzylamine and intermediate 3 gives, after recrystallisation from acetone, the title compound as white crystals m.p.: 275-279° C. Analysis for C₂₈H₂₅N₃O₃: Calculated: C,74.48;H,5.58;N,9.31;

Found:C,74.53;H,5.60;N,9.20%.

### EXAMLPLE 33

### Cis-2,3,6,7,12,12a-Hexahydro-6-(3-Methoxyphenyl)-2-Methyl-Pyrazino(2′,1′:6,1)Pyrido(3,4-b)Indole-1,4-Dione

The same two-step procedure but starting from methylamine and intermediate 5 gives, after recrystallisation from methanol, the title compound as white crystals m.p.: 267-269° C. Analysis for C₂₂H₂₁N₃O₃: Calculated: C,70.38;H,5.64;N,11.19; Found:C,70.32;H,5.59;N,11.25%.

### EXAMPLE 34

### Cis-2,3,6,7,12,12a-Hexahydro-6-(4-Ethoxyphenyl)-2-Methylpyrazino(2′,1′:6,1)Pyrido(3,4-b)Indole-1,4-Dione

The same two-step procedure but starting from methylamine and intermediate 6 gives, after recrystallisation from methanol, the title compound as white crystals m.p. :247-248° C. Analysis for C₂₃H₂₃N₃O₃: Calculated: C,70.93,H,5.95;N,10.79; Found:C,71.23;H,5.95;N,10.63%.

### EXAMIPLE 35

### Cis-2,3,6,7,12,12a-Hexahydro-6-(4-Ethoxyphenyl)-2-Cyclopropylmethylprazino(2′,1′:6,1)Pyrido(3,4-b)Indole-1,4-Dione

The same two-step procedure but starting from cyclopropylmethylamine and intermediate 6 gives, after recrystallisation from 2-propanol, the title compound as white crystals m.p.: 160-162° C. Analysis for C₂₆H₂₇N₃O₃: Calculated: C,72.71 ;H,6.34;N,9.78; Found:C,72.28;H,6.39;N,9.71%.

### EXAMPLE 36

### Cis-2,3,6,7,1 2,12a-Hexahydro-6-(2,3-Dihydrobenzo)Furan-5-yl)-2-Methylpyrazino(2′,1′:6,1)Pyrido(3,4-b)Indole-1,4-Dione

The same two-step procedure but starting from methylamine and intermediate 8 gives, after recrystallisation from methanol, the title compound as white crystals m.p. : 292-294° C. Analysis for C₂₃H₂₁)N₃O₃: Calculated: C,71.30;H,5.46;N,10.85; Found:C,71.15;H,5.56;N,10.84%.

### EXAMPLE 37

### Cis-2,3,6,7,12,12a-Hexahydro-6-(2,3-Dihydrobenzo(b)Furan-5-yl)-2-Cyclopropylmethyl-Pyrazino(2′,1′:6,1)Pyrido(3,4-b)Indole-1,4-Dione

The same two-step procedure but startmg from cyclopropylmethylamme and intermediate 8 gives, after recrystallisation from methanol, the title compound as white crystals m.p.: 165-166° C. Analysis for C₂₆H₂₅N₃O₃: Calculated: C,73.05;H,5.89;N,9.83; Found:C,73.08;H,5.97;N,9.87%.

### EXAMPLE 38

### Cis-2,3,6,7,12,12a-Hexahydro-6-(3,4-Ethylenedioxyphenyl)-2-Methylpyrazino(2′,1′:6,1)Pyrido(3,4-b)Indole-1,4-Dione

The same two-step procedure but starting from methylamine and intermediate 10 gives, after recrystallisation from acetone, the title compound as white crystals m.p.: 303-305° C. Analysis for C₂₃H₂₁)N₃O₄: Calculated: C,68.47;H,5.25;N,10.42; Found:C,68.35;H,5.31 ;N,10.27%.

### EXAMPLE 39

### Cis-2,3,6,7,12,12a-Hexahydro-6-(3,4-Ethylenedioxyphenyl)-2-Cyclopropyl Methylprazino(2′,1′:6,1)Pyrido(3,4-b)Indole-1,4-Dione

The same two-step procedure but starting from cyclopropylmethylamine and intermediate 10 gives, after recrystallisation from dichloromethanelether, the title compound as white crystals m.p.: 288-290° C. Analysis for C₂₆H₂₅N₃O₄: Calculated: C,70.41 ;H,5.68;N,9.47; Found:C,70.15;H,5.62;N,9.30%.

### EXAMPLE 40

### Cis-2,3,6,7,12,12a-Hexahydro-2-Butyl-6-(2-Chlorophenyl) Pyrazino(2′,1′:6,1)Pyrido(3,4-b)Indole-1,4-Dione

The same two-step procedure but starting from butylamine and intermediate 12 gives, after recrystallisation from methanol/water, the title compound as white crystals m.p.: 146° C. Analysis for C₂₄H₂₄ClN₃O₂(0.75 H₂O): Calculated: C,66.20;H,5.90;N,9.65; Found:C,66. 1 5;H,5.95;N,9.69%.

### EXAiMPLE 41

### Cis-2,3,6,7,12,12a-Hexahydro-6-(4-Chlorophenyl)-2-Methylpyrazino(2′,1′:6,1)Pyrido(3,4-b)Indole-1,4-Dione

The same two-step procedure but starting from methylamine and intermediate 13 gives, after recrystallisation from methanol, the title compound as white crystals m.p. : 274° C. Analysis for C₂₁H₁₈ClN₃O₂ (0.25 H₂O): Calculated: C,65.63;H,4.85;N,10.93; Found:C,65.39;H,4.84;N,10.85%.

### EXAMPLE 42

### Cis-2,3,6,7,12,12a-Hexahydro-2-Butyl-6-(4-Chlorophenyl)Pyrazino(2′,1′:6,1)Pyrido(3,4-b)Indole-1,4-Dione

The same two-step procedure but starting from butylamine and intermediate 13 gives, after recrystallisation from ethanouwater, the title compound as white crystals m.p.: 164-166° C. Analysis for C₂₄H₂₄ClN₃O₂: Calculated: C,68.32;H,5.73;CI,8.40;N,9.96; Found:C,68.48;H,5.64;Cl,8.37;N,9.99%.

### EXAMPLE 43

### Cis-2,3,6,7,12,12a-Hexahydro-6-(3,4-Dichlorophenyl)-2-Methylpyrazino(2′,1′:6,1)Pyrido(3,4-b)Indole-1,4-Dione

The same two-step procedure but starting from methylamine and intermediate 15 gives, after recrystallisation from ethanol/DMF, the title compound as white crystals m.p.: >260° C. Analysis for C₂₁H₁₇Cl₂N₃O₂ (0.5 H₂O): Calculated: C,59.39;H,4.29;N,9.93; Found:C,59.32;H,4. 1 6;N,9.99%.

### EXAMPLE 44

### Cis-2,3,6,7,12,12a-Hexahydro-2-Butyl-6-Phenyl-Pyrazino(2′,1′:6,1)Pyrido(3,4-Blindole-1,4-Dione

The same two-step procedure but starting from butylamine and cis-methyl 1,2,3,4-tetrahydro-1-phenyl-9H-pyrido(3,4-b)indole-3-carboxylatel) gives, after recrystallisation from methanol/water, the title compound as white crystals m.p.: 243-245° C. Analysis for C₂₄H25N₃O₂: Calculated: C,74.39;H,6.50;N,10.84; Found:C,74.54;H,6.51;N,I0.86%. 1. D. Soerens et al., J. Org. Chem. 44, 535 - 545 (1979).

### EXAiMPLE 45

### Cis-2,3,6,7,12,12a-Hexahydro-2-Benzyl-6-Phenyl-Pyrazino(2′,1′:6,1)Pyrido(3,4-Blindole-1,4-Dione

The same two-step procedure but starting from benzylamine and cis-methyl-1,2,3,4-tetrahydro-1-phenyl-9H-pyrido(3,4-b)indole-3-carboxy late gives, after recrystallisation from methanol, the title compound as white crystals m.p.: 1993-195° C. Analysis for C₂₇H2₃N₃O₂: Calculated: C,76.94;H,5.50;N,9.97; Found:C,77.23 ;H,5.54;N,9.97%.

### EXALMPLE 46

### Trans-2,3,6,7,12,12a-Hexahydro-2-Benzyl-6-Phenyl-Pyrazino(2′,1′:6,1)Pyrido(3,4-b)Indole-1,4-Dione

The same two-step procedure but starting from benzylamine and cis-methyl-1,2,3,4-tetrahydro-1-phenyl-9H-pyrido(3,4-b)indole-3-carboxy late gives, after recrystallisation from methanol, the title compound as white crystals m.p.: 284° C. Analysis for C₂₇H₂₃N₃O₂: Calculated: C,76.94;H,5.50;N,9.97; Found:C,76.88;H,5.45;N,9.89%.

### EXAMPLE 47

### Cis-2,3,6,7,12,12a-Hexahydro-2-Methyl-6-(1,2,3,4-Tetrahydro-6-Naphthyl)Pyrazino(2′,1′:6,1)Pyrido(3,4-b)Indole-1,4-Dione

The same two-step procedure but starting from methylamine and intermediate 17 gives, after recrystaUisation from methanol, the title compound as white crystals m. p. : 260° C. Analysis for C₂₅H₂₅N₃O₂: Calculated: C,75.16;H,6.31;N,10.52; Found:C,74.93;H,6.43;N,10.63%.

### EXAMPLE 48

### Cis-2,3,6,7,12,12a-Hexahydro-2-Isopropyl-6-(1,2,3,4-Tetrahydro-6-Naphthyl)Pyrazino(2′,1′:6,1)Pyrido(3,4-b)Indole-1,4-Dione

The same two-step procedure but starting from isopropylamine and intermediate 17 gives, after recrystallisation from the title compound as off-white crystals m.p.: 244-246° C. Analysis for C₂₇H₂₉N₃O₂ (0.25H₂O): Calculated: C,75.06;H,6.88;N,9.73; Found:C,75.00;H,6.83 ;N,9.69%.

### EXAMPLE 49

### Cis-2,3,6,7,12,12a-Hexahydro-2-CyclopTopylmethyl-6-(1,2,3,4-Tetrahydro-6-Naphthyl)-Pyrazino(2′,1′:6,1)Pyrido(3,4-b)Indole-1,4-Dione

The same two-step procedure but starting from cyclopropylmethylainine and intermediate 17 gives, after recrystallisation from ethanolipentane, the title compound as white crystals m.p. :125° C. Analysis for C₂₈H₂₉N₃O₂ (0.25 H₂O): Calculated: C,75.73 ;H,6.70;N,9.46; Found:C,75.45;H,6.86;N,9.14%.

### EXAMPLE 50

### Cis-2,3,6,7,12,12a-Hexahydro-2-Methyl-6-(2-Naphthyl)Pyrazino(2′,1′:6,1)Pyrido(3,4-b)Indole-1,4-Dione

The same two-step procedure but starting from methylamine and intermediate 18 gives, after recrystallisation from dichloromethane/methanol, the title compound as white crystals m.p. :>260° C. Analysis for C₂₅H21)N₃O₂ (0.25H₂O): Calculated: C,75.08;H,5.42;N,10.51; Found:C,75.35;H,5.42;N,10.49%.

### EXAMPLE 51

### Cis-2,3,6,7,12,12a-Hexahydro-2-Butyl-6-(2-Thienyl)-Pyrazino(2′,1′:6,1)Pyrido(3,4-Blindole-1,4-Dione

The same two-step procedure but starting from butylamine and intermediate 20 gives, after recrystallisation from ethanol, the title compound as white crystals m.p.: 226° C. Analysis for C₂₂H₂₃N₃O₂S: Calculated: C,67.15;H,5.89;N, 10.68; Found:C,67.39;H,5.88;N, 10.77%.

### EXAMPLE 52

### Cis-2,3,6,7,12,12a-Hexahydro-6-(5-Bromo-2-Thienyl)-2-Methylpyrazino(2′,1′:6,1)Pyrido(3,4-b)Indole-1,4-Dione

The same two-step procedure but starting from methylamine and intermediate 24 gives, after recrystallisation from ethanol, the title compound as a cream powder m.p. : 258° C. Analysis for C₁₉H₁₆BrN₃O₂S: Calculated: C,53.03;H,3.75;N,9.76; Found:C,53.01 ;H,3.78;N,9.69%.

### EXAMPLE 53

### Cis-2,3,6,7,12,12a-Hexahydro-6-(4-Bromo-2-Thienyl)-2-Methylpyrazino(2′,1′:6,1)Pyrido(3,4-b)Indole-1,4-Dione

The same two-step procedure but starting from methylamine and intermediate 26 gives, after recrystallisation from ethanol, the title compound as white crystals mp.: 292° C. Analysis for C₁₉H₁₆BrN₃O₂S (0.25H₂O): Calculated: C,52.48;H,3.82;N,9.66; Found:C,52.46;H,3.81 ;N,9.60%.

### EXAMPLE 54

### Cis-2,3,6,7,12,12a-Hexahydro-6-(5-Bromo-2-Thienyl)-2-Cyclopropylmethylpyrazino(2′,1′:6,1)Pyrido(3,4-b)Indole-1,4-Dione

The same two-step procedure but starting from cyclopropylmethylamine and intermediate 24 gives, after recrystallisation from ethanol, the title compound as white crystals m.p.: 190° C. Analysis for C2₂H₂₀BrN₃O₂S: Calculated: C,56.18;H,4.29;N,8.93; Found:C,55.92;H,4.28;N,8.74%.

### EXAIMPLE 55

### Cis-2,3,6,7,12,12a-Hexahydro-6-(5-Bromo-2-mbienyl)-2-Cyclopentylpyrazino(2′,1′:6,1)Pyrido(3,4-b)Indole-1,4-Dione

The same two-step procedure but starting from cyclopentylamine and intermediate 24 gives, after recrystallisation from ethanol, the title compound as white crystals m.p.: 252° C. Analysis for C₂₃H₂₂BrN₃O₂S: Calculated: C,57.03;H,4.58;N,8.67; Found:C,56.87;H,4.66;N,8.68%.

### EXAMPLE 56

### Cis-2,3,6,7,12,12a-Hexahydro-2-Methyl-6-(5-Methyl-2-Thienyl)Pyrazino(2′,1′:6,1)Pyrido(3,4-b)Indole-1,4-Dione

The same two-step procedure but starting from methylamine and the cis isomer of intermediate 66 gives, after recrystallisation from ethanol, the title compound as white crystals m.p.: 282° C. Analysis for C₂₀H₁₉N₃O₂S (0.25H₂O): Calculated: C,64.93;H,5.3 1;N,11.36; Found:C,64.84;H,5.28;N,10.81%.

### EXAMPLE 57

### Cis-2,3,6,7,12,12a-Hexahydro-2-Methyl-6-(3-Thienyl) Pyrazino(2′,1′:6,1)Pyrido(3,4-b)Indole-1,4-Dione

The same two-step procedure but starting from methylamine and intermediate 22 gives, after recrystallisation from acetone, the title compound as white crystals m.p.: 290-295° C. Analysis for C₁₉H₁₇N₃O₂S: Calculated: C,64.94;H,4.88;N, 11.96; Found: C, 64.81; H,4.95 ; N,11.68%.

### EXAMPLE 58

### Cis-2,3,6,7,12,12a-Hexahydro-2-Butyl-6-(3-Thienyl)-Pyrazino(2′,1′:6,1)Pyrido(3,4-b)Indole-1,4-Dione

The same two-step procedure but starting from butylamine and intermediate -22 gives, after recrystallisation from methanol, the title compound as white crystals m.p. : 236-239° C. Analysis for C2₂H2₃N₃O₂S: Calculated: C,67.15;H,5.89;N,10.68;S,8.15; Found:C,67.42;H,5.76;N, I D.57;S,8.01%.

### EXAMPLE 59

### Cis-2,3,6,7,12,12a-Hexahydro-2-Methyl-6-(3-Furyl)-Pyrazino(2′,1′:6,1)Pyrido(3,4-b)Indole-1,4-Dione

The same two-step procedure but starting from methylamine and the cis isomer of intermediate 28 gives, after recrystallisation from ether, the title compound as a white solid m.p. :250° C. Analysis for C₁₉H₁₇N₃O₃ (0.5H₂O): Calculated: C,66.27;H,5.27;N,12.20; Found:C,66.33;H,5.48 ;N,12.02%.

### EXAMPLE 60

### Cis-2,3,6,7,12,12a-Hexahydro-2-Methyl-6-(5-Methyl-2-Furyl)-Pyrazino(2′,1′:6,1)Pyrido(3,4-b)Indole-1,4-Dione

The same two-step procedure but starting from methylamine and intermediate 29 gives, after recrystallisation from ethanol, the title compound as a cream powder m.p.: 303° C. Analysis for C₂₀H₁₉N₃O₃ (0.25H₂O): Calculated: C,67.88;H,5.55;N,11.87; Found:C,67.90;H,5.50;N,11.98%.

### EXAMPLE 61

### Cis-2,3,6,7,12,12a-Hexahydro-2-Methyl-6-(4-Methylphenyl)-Pyrazino(2′,1′:6,1)Pyrido(3,4-b)Indole-1,4-Dione

The same two-step procedure but starting from methylamine and intermediate 31 gives, after recrystallisation from ethanol, the title compound as white crystals m.p.:>260° C. Analysis for C2_{2H21})N₃O₂ (0.25 H₂O): Calculated: C,72.61 ;H,5.95;N,l 1.55; Found:C,72.73;H,5.96;N,l 1.59%.

### EXAMPLE 62

### Cis-2,3,6,7,12,12a-Hexahydro-2-Isopropyl-6-(4-Methylphenyl)-Pyrazino(2′,1′:6,1)Pyrido(3,4-b)Indole-1,4-Dione

The same two-step-procedure but starting from isopropylamine and intermediate 31 gives, after recrystallisation from the title compound as white crystals m.p.: 170° C. Analysis for C2₄H₂₅₁N₃O₂ (0.5H₂O): Calculated: C,72.70;H,6.61;N,10.60; Found:C,73.06;H,6.43;N,9.66%.

### EXAMPLE 63

### Cis-2,3,6,7,12,12a-Hexahydro-2-Butyl-6-(4-Methylphenyl)-Pyrazino(2′,1′:6,1)Pyrido(3,4-b)Indole- 1,4-Dione

The same two-step procedure but starting from butylamine and intermediate 31 gives, after recrystallisation from methanol, the title compound as white crystals m.p.: 194° C. Analysis for C₂₅H₂₇N₃O₂ (0.5H₂O): Calculated: C,73. 15S;H,6.87;N,10.24; Found: C,73.01;H,6.84.N,10.26%.

### EXAMPLE 64

### Cis-2,3,6,7,12,12a-Hexahydro-2-Cyclopropylmethyl-6-(4-Methylphenyl)-Pyrazino(2′,1′:6,1)Pyrido(3,4-b)Indol-1,4-Dione

The same two-step procedure but starting from cyclopropylmethylamine and intermediate 31 gives, after recrystallisation from methanol/water, the title compound as white crystals m.p. :194° C. Analysis for C₂₅H₂₅N₃O₂ (1.1 H₂O): Calculated: C,71.61;H,6.54;N,10.02; Found:C,71.42.H,6.07;N,9.95%.

### EXAMPLE 65

### Cis-2,3,6,7,12,12a-Hexahydro-2-Methyl-6-(3-Methylphenyl)-Pyrazino(2′,1′:6,1)Pyrido(3,4-b)Indole- b 1,4-Dione

The same two-step procedure but starting from methylamine and intermediate 33 gives, after recrystallisation from ethanol, the title compound as white crystals m.p.: >260° C. Analysis for C₂₂H₂₁)N₃O₂; Calculated: C,73.52;H,5.89;N, 11.69; Found:C,73.60;H,5.97;N, 11.66%.

### EXAMPLE 66

### Cis-2,3,6,7,12,12a-Hexahydro-2-Butyl-6-(4-Trifluoromethylphenyl)-Pyrazino(2′,1′:6,1)Pyrido(3,4-b)Indole-1,4-Dione

The same two-step procedure but starting from butylamine and intermediate 35 gives, after recrystallisation from methanol/water, the title compound as white crystals m.p.: 155° C. Analysis for C₂₅H₂₄F₃N₃O₂ (0.5H₂O): Calculated: C,64.65;H,5.43;N,9.05; Found:C,64.78;H,5.40;N,9.01%.

### EXAMPLE 67

### Cis-2,3,6,7,12,12a-Hexahydro-2-Methyl-6-(4-Trifluoromethoxyphenyl)-Pyrazino(2′,1′:6,1)Pyrido(3,4-b)Indole-1,4-Dione

The same two-step procedure but starting from methylamine and the cis isomer of intermediate 65 gives, after recrystallisation from methanol, the title compound as white crystals m.p.:174-180° C. Analysis for C₂₂H₁₈F₃N₃O₃ (0.5H₂O): Calculated: C,60.27;H,4.37;N,9.58; Found:C,60.24;H,4.28 ;N,9.50%.

### EXAMPLE 68

### Cis-2,3,6,7,12,12a-Hexahydro-2-Methyl-6-(4-Hydroxyphenyl)-Pyrazino(2′,1′:6,1)Pyrido(3,4b)Indole-1,4-Dione

The same two-step procedure but starting from methylamnine and intermediate 39 gives, after recrystallisation from methanol, the title compound as yellow crystals m.p. :179-180° C. Analysis for C₂₁H₁₉N₃O₃(1.25H₂O): Calculated: C,65.70;H,5.64;N,10.94; Found:C,65.46;H,5.45;N, 10.92%.

### EXAiMPLE 69

### Cis-2,3,6,7,12,12a-Hexahydro-6-(3-Hydroxy-4-Methoxyphenyl)-2-Methylpyrazino(2′,1′:6,1)Pyrido(3,4-b)Indole-1,4-Dione

The same two-step procedure but starting from methylamine and intermediate 40 gives, after recrystallisation from ethanol, the title compound as white crystals m.p.:320° C. Analysis for C₂₂H₂₁N₃O₄(0.25H₂O): Calculated: C,66.74;H,5.47;N,10.61; Found:C,66.72;H,5.46;N,1 0.53%.

### EXAMPLE 70

### Cis-2,3,6,7,12,12a-Hexalhydro-6-(4-Hydroxy-3-Methoxyphenyl)-2-Methylpyrazino(2′,1′:6,1)Pyrido(3,4-b)Indole- 1,4-Dione

The same two-step procedure but starting from methylaine and intermediate 41 gives, after recrystallisation from dichloromethane/ethanol, the title compound as yellow crystals m.p.:264-265° C. Analysis for C₂₂H₂₁N₃O₄: Calculated: C,67.51;H,5.41;N,10.74; Found:C,67.05;H,5.41 ;N,10.62%.

### EXAMPLE 71

### Cis-2,3,6,7,12,12a-Hexahydro-2-Butyl-6-(4-Cyanophenyl)-Pyrazino(2′,1′:6,1)Pyrido(3,4-b)Indole-1,4-Dione

The same two-step procedure but starting from butylamine and intermediate 37 gives, after recrystallisation from methanol/water, the title compound as white crystals m.p.: 246° C. Analysis for C₂₅H₂₄N₄O₂ (1H₂O): Calculated: C,69.75;H,6.09;N,13.01; Found:C,69.50;H,5.96;N,12.86%.

### EXAMPLE 72

### Cis-2,3,6,7,12,12a-Hexahydro-6-(4-Ethylphenyl)-2-Isopropylpyrazino(2′,1′:6,1)Pyrido(3,4-b)Indole-1,4-Dione

The same two-step procedure but starting from isopropylamine and the cis isomer of intermediate 42 gives, after recrystallisation from n-pentane, the title compound as white crystals m.p.: 130° C. Analysis for C₂₅H₂₇N₃O₂ (0.5 H₂O): Calculated: C,73.15;H,6.87;N,10.24; Found:C,73.39;H,7.08;N,9.81%.

### EXAMPLE 73

### Cis-2,3,6,7,12,12a-Hexahydro-6-(4-Ethylphenyl)-2-Cyclopropyymethylpyrazino(2′,1′:6,1)Pyrido(3,4-b)Indole-1,4-Dione

The same two-step procedure but starting from cyclopropylmethylamine and the cis isomer of intermediate 42 gives, after recrystallisation from ethanol, the title compound as white crystals m.p.: 160° C. Analysis for C₂₆H₂₇N₃O₂: Calculated: C,75.52;H,6.58;N,10. 16; Found:C,75.54;H,6.62;N,10.08%.

### EXAMPLE 74

### Cis-2,3,6,7,12,12a-Hexahydro-6-(4-Isopropylphenyl)-2-Methylpyrazino(2′,1′:6,1)Pyrido(3,4-b)Indole-1,4-Dione

The same two-step procedure but starting from methylamine and intermediate 43 gives, after recrystallisation from ethanol, the title compound as white crystals m.p.: 244° C. Analysis for C₂₄H₂₅N₃O₂: Calculated: C,74.39;H,6.50;N,10.84; Found:C,74.27;H,6.53;N,11.05%.

### EXAMPLE 75

### Cis-2,3,6,7,12,12a-Hexahydro-2-Butyl-6-(4-Nitrophenyl)-Pyrazino(2′,1′:6,1)Pyrido(3,4-b)Indole-1,4-Dione

The same two-step procedure but starting from butylamine and intermediate 45 gives, after recrystallisation from methanol, the title compound as white crystals m.p.: 182° C. Analysis for C₂₄H₂₄N₄O₄ (0.25H₂O): Calculated: C,65.97;H,5.65;N,12.82; Found:C,65.92;H,5.62;N,12.96%.

### EXAMPLE 76

### Cis-2,3,6,7,12,12a-Hexahydro-6-(4-Dimethylaminophenyl)-2-Metylpyrazino(2′,1′:6,1)Pyrido(3,4-b)Indole-1,4-Dione

The same two-step procedure but starting from methylamine and the cis isomer of intermediate 47 gives after recrystallisation from methanol, the title compound as white crystals m.p.: 266° C. Analysis for C₂₃H₂₄N₄O₂: Calculated: C,71.1 ;H,6.23;N,14.42; Found:C, 71.19; H, 6.24 ; N, 14.34%.

EXAiMPLE 77

### Cis-2,3,6,7,112,12a-Hexahydro-2-Methyl-6-(3-Pyridal)-Pyrazino(2 ′,1′:6,1)Pyrido(3,4-b)Indole-1,4-Dione

The same two-step procedure but starting from methylamine and intermediate 48 gives after recrystallisation from chloroform, the title compound as white crystals m.p.:312° C. Analysis for C₂₀H18N₄O₂: Calculated: C,69.35;H,5.24;N,16.17; Found:C,69.08;H,5.20;N,16.19%.

### EXAMPLE 78

### (6R,12aR)-2,3,6,7,12,12a-Hexahydro-2-Methyl-6-(3,4)-Methylenedioxyphenyl)-Pyrazino(2′,1′:6,1)Pyrido(3,4b)Indole-1,4-Dione

(a) To a stirred solution of intermediate 54 (0.5 g) and NaHCO₃ (0.14 g) in anhydrous CHCl₃ (20 ml) is added dropwise chloroacetyl-chloride (0.27 ml) at 0° C. The resulting mixture is stirred for 1 hour at the same temperature and diluted with CHCl₃ (20 ml). Water (10 ml) is then added dropwise with stirring to the mixture, followed by a saturated solution of NaHCO₃. The organic layer is washed with water until neutrality and dried over Na₂SO₄. After evaporation of the solvent under reduced pressure, (6R,12aR)-methyl 1,2,3,4-tetrahydro-2-chloroacetyl-1-(3,4 methylenedioxyphenyl)-9H-pyrido(3,4-b)indole-3-carboxylate is obtained as an oil which is crystallised from ether to give a solid (0.38 g, m.p.: 233° C.) which is used without farther purification in the next step.

(b) To a stirred suspension of the chloroacetyl intermediate (0.37 g) in MeOH (20 ml) is added at room temperature a solution of methylamine (33% in EtOH) (0.4 ml), and the resulting mixture is heated at 50° C. under N₂ for 16 hours. The solvent is removed under reduced pressure and the residue is dissolved in CH₂Cl₂ (50 ml). After washing with water (3×20 ml), drying over Na₂SO₄ and evaporating to dryness, the residue is purified by flash chromatography eluting with CH₂Cl₂/MeOH (99/1) and recrystallised from 2-propanol to give the title compound as white crystals m.p. 302-303° C. Analysis for C22H₁₉N₃O₄: Calculated:C,67.86;H,4.92;N,10.79; Found:C,67.77;H,4.92;N,10.74%. 20° ((alpha))20° D+71.0° (C=1.00; CHCl₃).

The following compounds are obtained in a similar manner:

### EXAMPLE 79

### (6R,12aR)-2,3,6,7,12,12a-Hexahydro-2-Isopropyl-6-(3,4-Methylenedioxyphenyl)-Pyrazino(2′,1′:6,1)Pyrido(3,4-b)Indole-1,4-Dione

The same two-step procedure but starting from isopropylamine and intermediate 54 gives, after recrystallisation from methanol, the title compound as white crystals m.p.: 290-293° C. Analysis for C₂₄H₂₃N₃O₄: Calculated: C,69.05;H,5.55;N,10.07; Found:C,69.06;H,5.49;N,10.12%. ((alpha)) 20° D=+52.6° (C=1.14; CHCl₃).

### EXAMPLE 80

### (6R,12aR)-2,3,6,7,12,12a-Hexahydro-2-Butyl-6-(3,4-Methylenedioxyphenyl)-Pyrazino(2′,1′:6,1)Pyrido(3,4-b)Indole-1,4-Dione

The same two-step procedure but starting from butylamine and intermediate 54 gives, after recrystallisation from toluene/hexane, the title compound as white crystals m.p.: 209-210° C. Analysis for C₂₅H₂₅N₃O₄: Calculated: C,69.59;H,5.84;N,9.74; Found:C,69.70;H,5.93;N,9.74%. ((alpha))20°° D+50.2° (C=0.53; CHCl₃).

### EXAMPLE 81

### (6R,12aR)-2,3,6,7,12,12a-Hexahydro-2-Isobutyl-6-(3,4)-Methylenedioxyphenyl)-Pyrazino(2′,1′:6,1)Pyrido(3,4-b)Indole-1,4-Dione

The same two-step procedure but starting from isobutylarnine and intermediate 54 gives, after recrystallisation from methanol, the title compound as white crystals m.p.: 227-228° C. Analysis for C₂₅H₂₅N₃O₄: Calculated: C,69.59;H,5.84;N,9.74; Found:C,69.52;H,5. 87;N,9.74%. ((alpha))20° D =+45° (C=1.04; CHCl₃).

### EXAMPLE 82

### (6R,12aR)-2,3,6,7,12,12a-Hexahydro-2-Cyclopentyl-6-(3,4-Methylenedioxyphenyl-Pyrazino(2′,1′:6,1)Pyrido(3,4-b)Indole-1,4-Dione

The same two-step procedure but starting from cyclopentylamnine and intermediate 54 gives, after recrystallisation from ether, the title compound as white crystals m.p.: 237-239° C. Analysis for C₂₆H₂₅N₃O₄: Calculated: C,70.41 ;H,5.68;N,9.47; Found:C,70.13.H,5.67.N,9.42%. ((alpha))20° D=+36.6° (C=0.98; CHCl₃).

### EXAMPLE 83

### (6R,12aR)-2,3,6,7,12,12a-Hexahydro-6-(3,4-Methylenedioxyphenyl)-2-Cyclohexylmethyl-Pyrazino(2′,1′:6,1)Pyrido(3,4-b)Indole-1,4-Dione

The same two-step procedure but starting from cyclohexylmethylamine and the cis isomer of intermediate 56 gives, after recrystallisation from 2-prop anol the title compound as white crystals m.p. : 209° C. Analysis for C₂₈H₂₉N₃O₄: Calculated: C,71.32;H,6.20;N,8.91; Found:C,71.30;H,6.29;N,8.74%. ((alpha))20° D=+40.0° (C=0.99; CHCl₃).

### EXAMPLE 84

### (6R,12aR)-2,3,6,7,12,12a-Hexahydro-2-Cyclopropylmethyl-6-(4-Methoxyphenyl)-Pyrazino(2′,1′:6,1)Pyrido(3,4-b)Indole-1,4-Dione

The same two-step procedure but starting from cyclopropylmethylamine and intermediate 57 gives, after recrystallisation from methanol, the title compound as white crystals m.p.: 204-205° C. Analysis for C₂₅H₂₅N₃O₃(0.5H₂O): Calculated: C,70.74;H,6.17;N,9.90; Found:C,70.98;H,6.09;N,9.92%. ((alpha))20° D=+541° (C=1.03; CHCl₃).

### EXAMPLE 85

### (6R,12aR)-2,3,6,7,12,12a-Hexahydro-2-Butyl-6-(4-Methoxyphenyl)-Pyrazino(2′,1′:6,1)Pyrido(3,4-b)Indole-1,4-Dione

The same two-step procedure but starting from buylamine and intermediate 57 gives, after recrystallisation from 2-propanol, the title compound as white crystals m.p.: 183-184° C. Analysis for C₂₅H₂₇N₃O₃(0.5H₂O): Calculated: C,70.40;H,6.62;N,9.85; Found:C,70.55;H,6.64;N,9.92%. ((alpha))20° D=+45.4° (C=1.04; CHCl₃).

### EXAMPLE 86

### (6R,12aR)-2,3,6,7,12,12a-Hexahydro-2-Cyclopentyl-6-(4-Methoxyphenyl)-Pyrazino(2′,1′:6,1)Pyrido(3,4-b)Indole-1,4-Dione

The same two-step procedure but starting from cyclopentylamine and intermediate 57 gives, after recrystallisation from ether, the title compound as white crystals m.p.: 210-211° C. Analysis for C₂₆H₂₇N₃O₃; Calculated: C,72.71;H,6.34;N,9.78; Found:C,72.53;H,6.39;N,9.53%. ((alpha))20° D=+29.8° (C=1.07; CHCl₃).

### EXAMPLE 87

### (6R,12aR)-2,3,6,7,12,12a-Hexahydro-6-(3-Chloro-4-Methoxyphenyl)-2-Cyclopropylmethyl-Pyrazino(2′,1′:6,1)Pyrido(3,4-b)Indole-1,4-Dione

The same two-step procedure but starting from cyclopropylmethylamine and intermediate 59 gives, after recrystallisation from methanol, the title compound(underscore) as white crystals m.p.: 218-219° C. Analysis for C₂₅H2₄CIN₃0₃ (0.25 H₂0): Calculated: C,66.08;H,5.43;N,9.25; Cl, 7.80; Found: C, 66.11; H, 5.33; N, 9.03; Cl, 7.74%. ((alpha))20° D=+49.4° (C=1.03; CHCl₃).

### EXAMPLE 88

### (6R,12aR)-2,3,6,7,12,12a-Hexahydro-2-Cyclopentyl-6-(3-Chloro-4-Methoxyhenyl-Pyrazino(2′,1′:6,1)Pyrido(3,4-b)Indole-1,4-Dione

The same two-step procedure but starting from cyclopentylamine and intermediate 59 gives, after recrystallisation from methanol, the title compound as white crystals m.p.: 260-262° C. Analysis for C₂₆H₂₆ClN₃O₃: Calculated: C,67.31;H,5.65;Cl,7.64;N,9.06; Found:C,66.98;H,5.67;Cl,8.06;N,9.04%. ((alpha))20° D=+27.6° (C=1.05; CHCl₃).

### EXAMPLE 89

### (6R,12aR)-2,3,6,7,12,12a-Hexahydro-6-(3-Chloro-4-Methoxyphenyl)-2-Methylnyrazino(2′,1′:6,1)Pyrido(3,4-b)Indole-1,4-Dione

The same two-step procedure but starting from methylamine and intermediate 59 gives, after recrystallisation from methanol, the title compound as white crystals m.p. : 283-284° C. Analysis for C2₂H₂₀ClN₃O₃: Calculated: C,64.47;H,4.92;Cl,8.65;N,10.25; Found:C,64.49;H,4.92.C18.33.N,10.02%. ((alpha))20° D=+61.3° (0=1.00; CHCl₃).

### EXAMPLE 90

### (6R,12aR)-2,3,6,7,12,12a-Hexahydro-2-Isopropyl-6-(3-Chloro-4-Methoxyphenyol)-Pyrazino(2′,1′:6,1)Pyrido(3,4-b)Indole-1,4-Dione

The same two-step procedure but starting from isopropylamine and intermediate 59 gives, after recrystallisation from methanol, the title compound as white crystals m.p.: 302-304° C. Analysis for C₂₄H₂₄CIN₃O₃: Calculated: C,65.83 ;H,5.52;N,9.60; Found:C,65.83;H,5.57.N,9.73%. ((alpha))20° D=+39.8° (C=0.95; CHCl₃).

### EXAMPLE 91

### (6R,12aR)-2,3,6,7,12,12a-Hexahydro-6-(2,3-Dihydrobenzo(b)Furan-5-yl)-2-Methyl-Pyrazino(2′,1′:6,1)Pyrido(3,4-b)Indole-1,4-Dione

The same two-step procedure but starting from methylamine and intermediate 61 gives, after recrystallisation from dichloromethane/methanol, the title compound as white crystals m.p.: 288-291° C. Analysis for C₂₃H₂₁)N₃O₃: Calculated: C,71.30;H,5.46;N,10.85; Found:C,71.27;H,5.49;N,10.96%. ((alpha))20° D=+65.6° (C=0.4; CHCl₃).

### EXAMPLE 92

### (6R,12aR)-2,3,6,7,12,12a-Hexahydro-6-(2,3 -Dihydrobenzo(b)Furan-5-yl)-2-Methlcyclopropyl-Pyrazino(2′,1′:6,1)Pyrido(3,4-b)Indole-1,4-Dione

The same two-step procedure but starting from methylcyclopropylamine and intermediate 61 gives, after recrystallisation from methanol, the title compound as white crystals m.p.: 242-244° C. Analysis for C₂₆H₂₅N₃O₃: Calculated: C,73.05;H,5.89;N,9.83; Found:C,72.90;H,5 .93;N,9.98%. ((alpha))20° D=+55.4° (C=0.99; CHCl₃).

### EXAMPLE 93

### (6R,12aR)-2,3,6,7,12,12a-Hexahydro-6-(5-Indanyl)-2-Methylpyrazino (2′,1′:6,1)Pyrido(3,4-b)Indole -1,4-Dione

The same two-step procedure but starting from methylamine and intermediate 63 gives, after recrystallisation from methanol, the title compound as white crystals m.p.: 262° C. Analysis for C2₄H2₃N₃O₂: Calculated: C,74.78;H,6.01;N,10.90; Found:C,74.65;H,5.90;N,10.67%. ((alpha))20° D=+68.6° (C=0.98; CHCl₃).

### EXAMPLE 94

### (6R,12aR)-2,3,6,7,12,12a-Hexahydro-6-(5-Indanyl)-2-Cyclopropylmethylpyrazino(2′,1′:6,1)Pyrido(3,4-b)Indole-1,4-Dione

The same two-step procedure but starting from cyclopropylmethylamine and intermediate 63 gives, after recrystallisation fom methanol, the title compound as white crystals m.p. : 176° C. Analysis for C₂₇H₂₇N₃O₂ (0.25H₂O): Calculated: C,75.41; H, 6.45; N, 9.77; Found:C, 75.25; H, 6.51; N, 9.75%. ((alpha))20° D=+57.9° (C=1.00; CHCl₃).

### EXAMPLE 95

### (6R,12aR)-2,3,6,7,12,12a-Hexahydro-2-Methyl-6-(3,4-Methylenedioxyphenyl)-Pyrazino(2′,1′:6,1)Pyrido(3,4-b)Indole-1,4-Dione

To a stirred suspension of Intermediate 73 (12.5 g) in MeOH (400 ml) is added at room temperature a solution of methylamine (33% in EtOH) (13.7 ml) and the resulting mixture is heated at 50° C. under N₂ for 14 hours. The solvent is removed under reduced pressure and the residue is dissolved in CH₂Cl₂ (11). After washing with water (3×500 ml), drying over Na₂SO₄ and evaporating to dryness, the white solid obtained is recrystallised from 2-propanol to give the title compound as white needles.mp : 298-300° C. ((alpha))20° D=+71.3° (c=0.55, CHCl₃). Elemental analysis (C₂₂H₁₉N₃O₄) calculated: C, 67.86; H, 4.92; N, 10.79; found: C, 67.79; H, 4.95; N, 10.61%.

### EXAMPLE 96

### Cis-2,3,6,7,12,12a-Hexahydro-2,10-Dimethyl-6-(3,4-Methylenedioxyphenyl)-Pyrazino(2′,1′:6,1)Pyrido(3,4-b)Indole-1,4-Dione

The same two-step procedure as used to prepare Example 1, but starting from methylamine and the cis isomer of Intermediate 74, gives after recrystallisation from ethanol, the title compound as white crystals m.p. : 275° C. Analysis for C₂₃H₂₁N₃O₄ (0.4H₂O): Calculated: C, 67.27 ; H, 5.35 ; N, 10.23; Found: C, 67.36 ; H, 5.21; N, 10.31%.

### EXAMPLE 97

### (6R,12aR)-2,3,6,7,12,12a-Hexahydro-2-(3,4-Dimethoxybenzyl)-6-(3,4Methylenedioxyphenyl)-Pyrazino(2′,1′:6,1)Pyrido(3,4-b)Indole-1,4-Dione

The same two-step procedure as used to prepare Example 78, but starting from veratrylamine and intermediate 54 gives, after recrystallisation from methanol, the title compound as white crystals m.p. : 224-226° C. Analysis for C₃₀H₂₇N₃O₆: Calculated: C,68.56; H,5.18; N,8.00; Found: C,68.80; H,5.11; N,8.06%. ((alpha))20° D=+43.9° (C=1.02; CHCl₃).

### EXAMPLE 98

### Cis-2,3,6,7,12,12a-Hexahydro-6-(4-Aminophenyl)-2-Butyloprazino(2′,1′:6,1)Pyido(3,4-b)Indole-1,4-Dione

To a solution of Example 75 (1.5 g) in methanol (100 ml) is added SnCl₂.H₂O (3.06)and the resulting mixture is heated at reflux for 8 hours. The mixture is cooled to ambient temperature, poured into ice and is adjusted to pH5 with 1N NaOH. The methanol is evaporated off and the residue is basified to pH 11 with 1N NaOH and extracted with EtOAc (2×150 ml). After drying over Na₂SO₄ and evaporation of EtOAc, the resulting yellow powder is purified by radial chromatography eluting with CH₂Cl₂ to give the title compound as a white powder (550 mg) m.p.: 192° C. Analysis for C₂₄H₂₆N₄O₂ (1.3 H₂O): Calculated: C,67.68 ; H,6.77 ; N, 13.15; Found: C,67.74 ; H, 6.68 ; N, 13.02%.

### EXAMPLE 99

### Cis-2,3,6,7,12,12a-Hexahydro-6-(4-Acetamidophenyl)-2-Butylpyrazino(2′,1′:6,1)Pyrido(3,4-b)Indole-1,4-Dione

To a solution of Example 98 (0.2 g) in THF (15 ml) is added triethylamine (76 μL) andacetyl chloride (39 μL) and the resulting solution is stirred at room temperature for 2 hours. After evaporation of THF, the resulting residue is taken up in CH₂Cl₂ (100 ml), washed with water (2×50 ml) and dried over Na₂SO₄. After evaporation of CH₂Cl₂, the resulting solid is reciystallised from MeOH/H₂O to give the title compound as a cream powder (120 mg) m.p.: 246° C. Analysis for C₂₆H₂₈N₄O₃: Calculated: C,70.25; H,6.35; N,12.60; Found: C,69.85; H, 6.38 N,12.56%.

### EXAMPLE 100

### Cis-2,3,6,7,12,12a-Hexahydro-2-Butyl-6-(4-Methylsulfonamidophenyl)-Pyrazino(2′,1′:6,1)Pyrido(3,4-b)Indole-1,4-Dione

To a solution of Example 98 (0.2 g) in THF (5 ml) is added triethylamine (228 μL) andmethanesulfonyl chloride (126 4μL) and the solution is heated at reflux for 6 hours. After evaporation of THF, the residue is taken up in CH₂Cl₂, washed with water and dried over Na₂SO₄. After evaporation of CH₂Cl₂, the residue is purified by radial chromatography eluting with CH₂Cl₂/MeOH (95/5) to give the title compound as a brown powder (30 mg) m.p.: 188° C. Analysis for C₂₅H₂₈N₄O₄S (0.75 H₂O): Calculated: C,60.77; H,6.02; N,11.34; Found: C,60.61 ; H, 6.02; N,10.82%.

### EXAMLE 101

### (6R,12aR)-2,3,6,7,12,12a-Hexahydro-6-(3,4-Methylenedioxyphenyl)-Pyrazino(2′,1′:6,1)Pyrido(3,4-b)Indole-1,4-Dione

The same two-step procedure but starting from anmmonia and intermediate 54 gives, after recrystallisation from methanol, the title compound as white crystals m.p.: 285-290° C. Analysis for C₂₁H₁₇N₃O₄: Calculated: C, 67.19; H, 4.56; N, 11.19; Found: C, 67.30; H, 4.66; N, 11.11%. ((alpha))20°)D))=+88° (c=0.48; pyridine).

### EXAMPLE 102

### (6R,12aR)-2,3,6,7,12,12a-Hexahydro-6-(3,4-Methylenedioxyphenyl)-2-(2-Proponyl -Pyrazino(2′,1′:6,1)Pyrido(3,4-b)Indole-1,4-Dione

The same two-step procedure but starting from propargylanine and intermediate 54 gives, after recrystallisation from acetone, the title compound as white crystals m.p.: 271° C. Analysis for C₂₄H₁₉N₃O₄: Calculated: C, 69.72; H, 4.63 ;N, 10.16; Found: C, 69.95; H, 4.66; N, 10.06%. ((alpha))20°)D))=+51.70 (c=0.49 ; CHCl₃).

### EXAMPLE 103

### (6R,12aR)-2,3,6,7,12,12a-Hexahydro-2-(3,4-Methylendioxybenzyl)-6-(3,4-Methylenedioxyphenyl-Pyrazino(2′,1′:6,1)Pyrido(3,4-Indole-1,4-Dione

The same two-step procedure but starting-from piperonylamine and intermediate 54 gives, after recrystallisation from methanol, the title compound as white crystals m.p.: 204-206° C. Analysis for C₂₉H₂₃N₃O₆: Calculated: C, 68.36; H, 4.55 N, 8.25; Found: C, 68.25; H, 4.49; N, 8.41. ((alpha))20°)D))=+43° (c=1.01;CHCl₃).

### EXAMPLE 104

### (6R,12aR)-2,3,6,7,12,12a-Hexahydro-2-(3,4-Dimethoxyphenethyl)-6-(3,4-Methylenedioxyphenyl)-Pyrazino(2′,1′:6,1)Pyrido(3,4-b)Indole-1,4-Dione

The same two-step procedure but starting from 3,4-dimethoxyphenethylamine and intermediate 54 gives, after recrystallisation from dichloromethane/ether, the title compound as white crystals m.p.: 265-266° C. Analysis for C₃₁H₂₉N₃O₆: Calculated: C, 69.00; H, 5,42; N, 7.79; Found: C, 68.68; H, 5.35; N, 7.78%. ((alpha))20°)D)) =+38.30 (c=1.12 ; CHCl₃).

### EXAMPLE 105

### (6R,12aR)-2,3,6,7,12,12a-Hexahydro-2-Furfuryl-6-(3,4-Methylenedioxyphenyl)-Pyrazino(2′,1′:6,1)Pyrido(3,4-b)Indole-1,4-Dione

The same two-step procedure but starting from furfiurylamine and intermediate 54 gives, after recrystallisation from methanol, the title compound as white crystals m.p.: 219° C. Analysis for C₂₆H₂₁N₃O₅)): Calculated: C, 68.56 ; H, 4.65; N, 9.23 Found: C, 68.16 ; H, 4.63 ; N, 9.15%. ((alpha))20°)D))=+58.1° (c=1.2; CHCl₃)

### EXAMPLE 106

### (6R,12aR)-2,3,6,7,12,12a-Hexahydro-6-(3,4-Methylenedioxyphenyl)-2-(2-Thienylmethyl)-Pyrazino(2′,1′:6,1)Pyrido(3,4-b)Indole-1,4-Dione

The same two-step procedure but starting from 2-thiophenemethylamine and intermediate 54 gives, after recrystallisation from methanol/water, the title compound as white crystals m.p.: 155-157° C. Analysis for C_{26H21})N₃O₄S : Calculated: C, 66.23 H, 4.49 ;N, 8.91 ; S, 6.8 ; Found: C, 66.13 ; H, 4.54; N, 9.12 ;S, 6.78%. ((alpha))20°)D))=+70.4° (c=1.03 ; CHCl₃).

### EXAMPLE 107

### (6R,12aR)-2,3,6,7,12,12a-Hexahydro-6-(4-Methoxyphenyl)-2-Methyl-Pyrazino(2′,1′:6,1)Pyrido(3,4-b)Indole-1,4-Dione

The same two-step procedure but starting from methylamine and intermediate 57 gives, after recrystallisation from methanol, the title compound as white crystals m.p.: 285-288° C. Analysis for C₂₂H₂₁)N₃O₃ :Calculated: C, 70.38; H, 5.64; N, 11.19; Found: C, 70.31; H, 5.69; N, 11.29%. ((alpha))20°)D))=+59° (c=1.19; CHCl₃).

### EXAMPLE 108

### (6R,12aR)-2,3,6,7,12,12a-Hexahydro-2-Ethyl-6-(4-Methoxyphenyl)-Pyrazino(2′,1′:6,1)Pyrido(3,4-b)Indole-1,4-Dione

The same two-step procedure but starting from ethylarnine and intermediate 57 gives, after recrystallisation from methanol, the title compound as white crystals m.p.: 277° C. Analysis for C2₃H2₃N₃O₃: Calculated: C, 70.93; H, 5.95; N, 10.79; Found: C, 70.90; H, 5.96; N, 10.54%. ((alpha))20°)D))=+52° (c=1.28 ; CHCl₃).

### EXAMPLE 109

### (6R,12aR)-2,3,6,7,12,2a-Hexahydro-6-(7-(4-Methyl-3,4-Dihydro-2H-Benzo( 1,4) Oxazinyl))-2-Methyl-Pyrazino(2′,1′:6,1)Pyrido(3,4-b)Indole-1,4-Dione

The same two-step procedure but starting from intermediate 75 and methylamine gives, after recrystallisation from ethanol, the title compound as white crystals m.p.: 285-288° C. Analysis for C2₄H2₄N₄O₃ (0.5 H₂O) : Calculated: C, 67.75 ; H, 5.92 ; N, 13.17 ; Found: C, 68.02 ; H, 6.00 ; N, 13.18%. ((alpha))20°)D)) =+71.7° (c=1, pyridine).

### EXAMPLE 110

### (6R,12aR)-2,3,6,7,12,12a-Hexahydro-6-(5-(N-Benzylindolinyl))-2-Methylpyrazino(2′,1′:6,1)Pyrido(3,4b)Indole-1,4-Dione

The same two-step procedure but starting from intermediate 77 and methylamine gives, after recrystallisation from dichloromethane/methanol, the title compound as white crystals m.p.: 223-225° C. Analysis for C₃₀H₂₈N₄O₂: Calculated: C, 75.61; H, 5.92; N, 11.76; Found: C, 75.2; H, 5.78; N, 11.67%. ((alpha))20°)D)) =+20.4° (c=0.5, CHCl₃).

### EXAMPLE 111

### (6R,12aR)-2,3,6,7,12,12a-Hexahydro-6-(5-Indolinyl)-2-Methyl-Pyrazino(2′,1′:6,1)Pyrido(3,4-b)Indole-1,4-Dione

A solution of Example 110 (1.05 g , 2.2 mmol) in methanol (100 ml) is hydrogenated in the presence of 10% Pd-C (100 mg) for 48 hours at room temperature. After removal of the catalyst, the solvent is evaporated in vacuo to leave a residue which is purified by flash chromatography eluting with dichloromethane/methanol : 96/4. The solid obtained is recrystallised from dichloromethane/methanol to give the title compound (300 mg) as white crystals m.p.: 240° C. Analysis for C2₃H2₂N₄O₂ (0.5 H₂O): Calculated: C, 69.86 ; H, 5.86 ; N, 14.17; Found: C, 70.13; H, 5.77 ; N, 14.06%. ((alpha))20°)D))=+55.9° (c=1.18; pyridine).

### EXAMPLE 112

### Cis-2,3,6,7,12,12a-Hexahydro-6-(4-Ethylphenyl)-2-Methyl-Pyrazino(2′,1′:6,1)Pyrido(3,4-b)Indole-1,4-Dione

The same two-step procedure but starting from methylamine and the cis isomer of intermediate 42 gives, after recrystallisation from methanol, the title compound as white crystals m.p. 254° C. Analysis for C₂₃H₂₃N₃O₂ (0.25 H₂O): Calculated: C, 73.09; H, 6.27; N, 11.12; Found: C, 73.03 ; H, 6.18; N, 11.36%.

### EXAMPLE 113

### (6R,12aR)-2,3,6,7,12,12a-Hexahydro-6-(4-Carbomethoxyphenyl)-2-Methyl-Pyrazino(2′,1′:6,1)Pyrido(3,4-b)Indole-1,4-Dione

The same two-step procedure but starting from intermediate 78 (cis isomer) and methylamine gives, after recrystallisation from methanol, the title compound as white crystals m.p.: 308-312° C. Analysis for C2_{3H21})N₃O₄: Calculated: C, 68.47; H, 5.25; N, 10.42; Found: C, 68.76; H, 5.18; N, 10.35%. ((alpha))20°)D))=+97.7° (c=1, pyridine).

### EXAIMPLE 114

### (5aR,12k,14aR)-1,2,3,5a,6,11,12,14a-Octahydro-12-(3,4-Methylenedioxyphenyl)-Pyrrolo(1″,2″:4′,5′)Pyrazino(2′,1′:6,1)Pyrido(3,4-b)Indole-5-1,4-Dione

A solution of intermediate 80 (0.7 g, 1.2 mmol) in a mixture of methanol/THF (80/40 ml) is hydrogenated in the presence of 10% Pd-C (75 mg) for 48 hours at 40° C. After removal of the catalyst, the solvent is evaporated in vacuo to leave a residue, which is purified by flash chromatography eluting with dichloromethane/methanol : 98/2. The white solid obtained is recrystallised from methanol to give the title compound (180 mg) as white crystals m.p.: 284-287° C. Analysis for C2_{4H21})N₃O₄: Calculated: C, 69.39; H, 5.10; N, 10.11; Found: C, 69.47 ; H, 5.11 ; N, 9.97%. ((alpha))20°)D))=+21.7° (c=0.64, CHCl₃).

### EXAMPLE 115

### (5aR,12R,14aS)-1,2,3,5,6,11,12,14a-Octahydro-12-(3,4-Methylenedioxyphenyl)-Pyrrolo(1″,2″:4′,5′)Pyrazino(2′,1′:6,1)Pyrido(3,4-b)Indole-5-1,4-Dione

A solution of intermediate 81 (0.8 g, 1.37 =nol) in methanol (40 ml) is hydrogenated in the presence of 10% Pd-C (100 mg) for 5 hours at 45° C. After removol of the catalyst the solvent is evaporated in vacuo to leave a residue, which is purified by flash chromatography eluting with dichloromethane/methanol : 98/2. The solid obtained is recrystallised from methanol to give the title compound (300 mg) as white crystals m.p.:302-304° C. Analysis for C₂₄H₂₁)N₃O₄: Calculated: C, 69.39; H, 5.10;N, 10.11;Found:C,69.35;H,5.11 ;N, 10.10%. ((alpha))20°)D))=+106.8° (c=1.08, CHCl₃).

### EXAMPLE 116

### (3R, 6R, 12aR)-2,3,6,7,12,12a-Hexahydro-2,3-Dimethyl-6-(3,4-Methylenedioxyphenyl)-Pyrazino(2′,1′:6,1)Pyrido(3,4-b)Indole-1,4-Dione

To a stirred solution of intermediate 82 (0.15 g, 0.34 mmol) in THF (15 ml) is added at room temperature a solution of methylamine (33% in EtOH) (0.32 ml), and the resulting solution is heated at reflux under N₂ for 24 hours. The solvent is removed under reduced pressure, and the residue is dissolved in CH₂Cl₂ (25 ml). After washing with water (2×20 ml), drying over Na₂SO₄ and evaporating to dryness, the crude product is purified by flash chromatography eluting with dichloromethane/methanol: 99/1. The white solid obtained is recrystallised from methanol to give the title compound as white crystals (80 mg) m.p. : 219-220° C. Analysis for C₂₃H₂₁)N₃O₄: Calculated: C, 68.47 ; H, 5.25 ; N, 10.42 ; Found: C, 68.39; H, 5.21; N, 10.42%. ((alpha))20°)D))=+89.6° (c=1; CHCl₃).

### EXAMPLE 117

### (3S, 6R, 1 2aR)-2,3,6,7,12,12a-Hexahydro-2,3-Dimethyl-6-(3,4-Methylenedioxyphenyl)-Pyrazino(2′,1′:6,1)Pyrido(3,4-b)Indole-1,4-Dione

To a stirred solution of intermediate 83 (0.3 g, 0.68 mmol) in TBF (30 ml) is added at room temperature a solution of methylamine (33% in EtOH) (0.68 ml), and the resulting solution is treated at reflux under N₂ for 6 days. The solvent is removed under reduced pressure and the residue is dissolved in CH₂Cl₂ (50 ml). After washing with water (2,25 ml), drying over Na₂SO₄ and evaporating to dryness, the crude product is purified by flash chromatography eluting with dichloromethane/methanol: 99/1. The oily residue obtained is crystallised from methanol to give the title compound as white crystals (40 mg) m.p. :307-309° C. Analysis for C2_{3H21})N₃O₄: Calculated: C, 68.47 ; H, 5.25 ; N, 10.42; Found: C, 68.35; H, 5.33; N, 10.42%. ((alpha))20°)D))=+65.2° (c=1.15 ; CHC₃).

### EXAMPLE 118

### (6R, 12aR)-2,3,6,7,12,12a-Hexahydro-6-(3,4-Dihydroxyphenyl)-2-Methylpyrazino(2′,1′:6,1)Pyrido(3,4-b)Indole-1,4-Dione

A solution of intermediate 86 (0.75 g; 1.34 mmol) in a mixture of ethanol/THE (70/30ml) is hydrogenated in the presence of 10% Pd-C (75 mg) for 24 hours at room temperature. After removal of the catalyst, the solvent is evaporated in vacuo to leave a white solid which is recrystallisated from methanol to give the title compound (0.35 g) as white crystals m.p.: 224-226° C. Analysis for C₂₁H₁₉N₃O₄: Calculated: C, 66.83; H, 5.07; N, 11.13; Found: C, 66.58; H, 5.01; N, 11.04%. ((alpha))20°)D))=+58.4° (c=1.04; pyridine).

### EXAMPLE 119

### (6R,12aR)-2,3,6,7,12,12a-Hexahydro-2-Methyl-6-(5-(2-Methylisoindolinyl))Pyrazino(2′,1′:6,1)Pyrido(3,4-b)Indole-1,4-Dione

The same two-step procedure but starting from intermediate 87 and methylamine gives a crude oil which is purified by flash chromatography eluting with dichloromethane/methanol/triethylamine : 92/8/0.1%. The solid obtained is recrystallized from isopropanol/propyl ether/water to give the title compound (20 mg) as off-white crystals m.p.: 236° C. Analysis for C₂₄H₂₄N₄O₂ (2.68 H₂O) Calculated: C, 64.23 ; H, 6.59 ; N, 12.48 ; Found: C, 64.21; H, 6.43 ; N, 12.02%. ((alpha))20O)D))=+61 1°(c=0.5 ; CH₃OH).

### EXAMPLE 120

### (6R, 12aR)-2, 3, 6, 7, 12, 12a-Hexahydro-6-(5-Benzofuranyl)-2-Methyl-Pyrazino[2′,1′:6,1]Pyrido[3,4-b]Indole-1,4-Dione

To a stirred suspension of Intermediate 90 (0.42 g) in methanol (30 ml) is added at ambient temperature a solution of methylamine (33% in ETOH) (0.47 ml), and the resulting mixture is heated at 50° C. under N₂ for 72 hours. The solvent is removed under reduced pressure and dissolved in-dichloromethane. After washing with water, drying over Na₂SO₄ and evaporating to dryness, the crude product is purified by crystallization from methanol to give the title compound as white crystals (0.21 g). m.p.: 291-293° C.

Analysis for C₂₃H₁₉N₃O₃:

Calculated: C,71.68;H,4.97;N,10.90;

Reported:C,71.5;H,4.91 ;N,10.74%.

[a]20D +55.7′ (C=I; CHCI₃).

### EXAMPLE 121

### (6R, 12aR)-2, 3, 6, 7, 12, 12a-Hexahydro-6-(5-Benzofuranyl)-Pyrazino[2′,1′:6,1]Pyrido [3-4-b]Indole-1,4-Dione

The same procedure as employed in the preparation of Example 120 but starting from ammonia and Intermediate 3 gives, after recrystallization from methanol, the title compound as white crystals. m.p.: 310-311° C.

Analysis for C₂₂H₁₇N₃O₃ (0.4 MEOH):

Reported: C,70.03; H,4.88; N,10.94;

Found: C,70.01; H,4.8; N,10.61%;+60.4−(C=0.5;pyridine).

### EXAMPLE 122

### (6R, 12aR)-2, 3, 6, 7, 12, 12a-Hexahydro-6-(5-Benzofuranyl)-2-Isopropyl-Pyrazino [2′,1′:6,1]Pyrido [3,4-B]Indole-1,4-Dione

The same procedure as employed in the preparation of Example 120 but starting from isopropylamine and Intermediate 90 gave, after recrystallisation from methanol, the title compound as white crystals.

m.p.:291-292° C.

Analysis for C₂₅H₂₃N₃O₃ (0.6 MEOH):

Calculated: C,71.06; H,5.92; N,9.71;C,70.94; H,5.62; N,9.77%.

[a]20D−+37.9−(C=I; CHCI3)−

### EXAMPLE 123

### (3S, 6R, 12aR)-2, 3, 6, 7, 12, 12a-Hexahydro-6-(5-Benzofuranyl)-3-Methyl-Pyrazino[2′,1′:6,1]Pyrido [3,4-b]Indole-1,4-Dione

A solution of Intermediate 91 (0.3 g) in the presence of 10% Pd/C (30 mg) in methanol (10 ml) is stirred under an atmosphere of hydrogen at 50° C. for two hours. The reaction mixture is cooled, filtered through Celite, the filter cake washed with methanol and the filtrate evaporated in vacuo. The residue was purified by flash chromatography, eluting with dichloromethane/methanol (98/2) to give the title compound as white crystals after recrystallization from methanol (0.15 g).

M.P. : 150-151° C.

Analysis for C₂₃H₁₉N₃O₃ (0.1MeOH)

Calculated: C,71.39; H,5.03; N,10.81;

Found: C,71.08; H,5.16; N,10.50%; [a]′D=+50−(C=0.25; CHCl₃).

### EXAMPLE 124

### (3S, 6R, 12aR)-2, 3, 6, 7, 12, 12a-Hexahydro-6-(5-Benzofuranyl)-3-Methyl-Pyrazino[2′,1′: 6,1]Pyrido[3,4-b]Indole-1,4-Dione

The same procedure as employed in the preparation of Example 123 but starting from Intermediate 92 (0.52 g) and using 10% Pd/C (50 mg) in methanol (20 ml) gave, after recrystallization from methanol, the title compound as white crystals (40 mg).

m.p.: 323-324° C.

Analysis for C₂₄H₂₁ N₃O₃. (0.1 Methanol)

Calculated: C,71.52; H,5.35; N,10.43;

C,71.71; H,5.44; N,10.39%;

[ai20 C)=+53′ (C=0.35; CHCl3)−

It will be understood that various changes and modifications can be made in the details of procedure, formulation and use without departing from the spirit of the invention, especially as defined in the following claims.

We claim:

1. A method of treating a mammal having precancerous lesions comprising administering a pharmacologically effective amount of a compound of Formula I or pharmaceutically acceptable salt thereof: 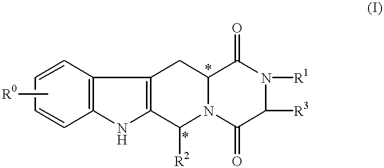
wherein R⁰ represents hydrogen, halogen or C1-6 alkyl;
R¹ represents hydrogen or C₁₋₆alkyl;
R² is benzofiran which may be optionally substituted by one or more groups selected from halogen and C₁₋₃ alkyl; and
R³ represents hydrogen or C₁₋₃alkyl.

2. The method of claim 1 wherein R⁰ represents hydrogen.

3. The method of claim 2 wherein R¹ is selected from hydrogen, methyl, and iso-propyl.

4. The method of claim 3 wherein R³ represents hydrogen or methyl.

5. The method of claim 1 wherein said compound is selected from the group consisting of (6R,12aR)-2,3,6,7,12,12a-Hexahydro-6-(5-benzofuranyl)-2-methyl-pyrazino [2′, 1′:6,1 lpyrdo[3,4-b]indole-1,4-dione; (6R, 12aR)-2,3,6,7,12,12a-Hexahydro-6-(5-benzofuranyl)-pyrazino[2′,1′:6,1] pyrido [3,4-b]indole-1,4-dione; (3S, 6R, 12aR)-2,3,6,7,12,12a-Hexahydro-6-(5-benzofiranyl)-3-methylpyrazino[2′,1′:6,1] pyrido [3,4-b]indole-1,4-dione; (3S, 6R, 12aR)-2,3,6,7,12,12a-Hexahydro-6-(5-benzofiranyl)-2,3-dimethyl-pyrazino[2′,1′:6,1] pyrido [3,4-b]indole-1,4-dione; and (6R,12aR)-2,3,6,7,12,12a-Hexahydro-6-(5-benzofiranyl)-2-isopropyl-pyrazino[2′,1′:6,1] pyrido [3,4-b]indole-1,4-dione; and physiologically acceptable solvates thereof.

6. The method of claim 1 wherein said compound is selected from (6R, 12aR)-2,3,6,7,12,12a-Hexahydro-6-(5-benzofuranyl)-2-methyl-pyrazino[2′,1′:6,1]pyrido[3,4-b]indole-1,4-dione and physiologically acceptable solvates thereof.

7. A method for inhibiting the growth of neoplastic cells comprising exposing the cells to a growth inhibiting effective amount of a compound of Formula I or pharmaceutically acceptable salt thereof: 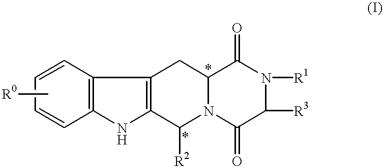
wherein R⁰ represents hydrogen, halogen or C₁₋₆ alkyl;
R¹ represents hydrogen or C₁₋₆alkyl;
R² is benzofuran which may be optionally substituted by one or more groups selected from halogen and C₁₋₃ alkyl; and
R³ represents hydrogen or C₁₋₃alkyl.

8. The method of claim 7 wherein R⁰ represents hydrogen.

9. The method of claim 8 wherein R¹ is selected from hydrogen, methyl, and iso-propyl.

10. The method of claim 9 wherein R³ represents hydrogen or methyl.

11. The method of claim 7 wherein said compound is selected from the group consisting of (6R,12aR)-2,3,6,7,12,12a-Hexahydro-6-(5-benzofuranyl)-2-methyl-pyrazino [2′, 1′:6,1 lpyrido[3,4-b]indole-1,4-dione; (6R, 12aR)-2,3,6,7,12,12a-Hexahydro-6-(5-benzofuranyl)-pyrazino[2′,1′:6,1] pyrido [3,4-b]indole-1,4-dione; (3S, 6R, 12aR)-2,3,6,7,12,12a-Hexahydro-6-(5-benzofuranyl)-3 -methylpyrazino[2′,1′:6,1] pyrido [3,4-b]indole-1,4-dione; (3S, 6R, 12aR)-2,3,6,7,12,12a-Hexahydro-6-(5-benzofuranyl)-2,3-dimethyl-pyrazino[2′,1′:6,1] pyrido [3,4-b]indole-1,4-dione; and (6R,12aR)-2,3,6,7,12,12a-Hexahydro-6-(5-benzofuranyl)-2-isopropyl-pyrazino[2′, 1′:6,1] pyrido [3,4-b]indole-1,4-dione; and physiologically acceptable solvates thereof.

12. The method of claim 7 wherein said compound is selected from (6R, 12aR)-2,3,6,7,12,12a-Hexahydro-6-(5-benzofiuranyl)-2-methyl-pyrazino[2′,1′:6,1]pyrido[3,4-b]indole-1,4-dione and physiologically acceptable solvates thereof.

13. A method for regulating apoptosis in human cells comprising exposing said cells to an effective amount of a compound of the formula: 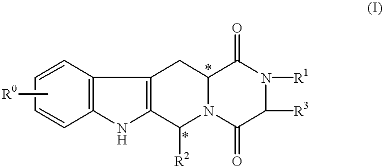
wherein R⁰ represents hydrogen, halogen or C¹⁻⁶ alkyl;
R¹ represents hydrogen or C1-6alkyl;
R² is benzofaran which may be optionally substituted by one or more groups selected from halogen and C₁₋₃ alkyl; and
R³ represents hydrogen or C₁₋₃alkyl.

14. The method of claim 13 wherein R⁰ represents hydrogen.

15. The method of claim 14 wherein R¹ is selected from hydrogen, methyl, and iso-propyl.

16. The method of claim 15 wherein R³ represents hydrogen or methyl.

17. The method of claim 13 wherein said compound is selected from the group consisting of (6R, 12aR)-2,3,6,7,12,12a-hexahydro-6-(5-benzofuranyl)-2-methyl-pyrazino [2′, 1′:6,1 lpyrido[3,4-b]indole-1,4-dione; (6R, 12aR)-2,3,6,7,12,12a-Hexahydro-6-(5-benzofuranyl)-pyrazino[2′,1′:6,1] pyrido [3 ,4-b]indole-1,4-dione; (3S, 6R, 12aR)-2,3,6,7,12, 12a-Hexahydro-6-(5-benzofiranyl)-3-methylpyrazino[2 ′,1′:6,1] pyrido [3,4b]indole-1,4-dione; (3S, 6R, 12aR)-2,3,6,7,12,12a-Hexahydro-6-(5-benzofuranyl)-2,3-dimethyl-pyrazino[2′,1′:6,1] pyrido [3,4-b]indole- 1,4-dione; and (6R, 1 2aR)-2,3,6,7,12,12a-Hexahydro-6-(5-benzofuranyl)-2-isopropyl-pyrazino[2′, 1′:6,1] pyrido [3,4-b]indole-1,4-dione; and physiologically acceptable solvates thereof.

18. The method of claim 13 wherein said compound is selected from (6R, 12aR)-2,3,6,7,12,12a-Hexahydro-6-(5-benzofuranyl)-2-methyl-pyrazino[2′,1′:6,1]pyrido[3,4-b]indole-1,4-dione and physiologically acceptable solvates thereof.

19. A method of treating a mammal having precancerous lesions comprising administering a pharmacologically effective amount of a compound of Formula I or pharmaceutically acceptable salt thereof: 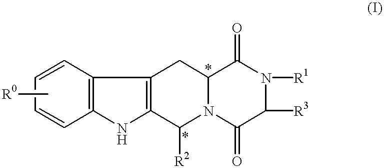
wherein R⁰ represents hydrogen, halogen or C1-6 alky;
R¹ represents hydrogen, C1-6 alkyl, C₂₋₆alkenyl, C₂₋₆ alynyl, haloC1-6 alkyl, C₃₋₈cycloalkyl, C₃₋₈-cycloalkylC₁₋₃alkyl, arylC1-3alkyl or heteroarylC₁₋₃alkyl, where aryl means phenyl or phenyl substituted by one or more (e.g. 1, 2 or 3) substituents selected from halogen, C1-6alkyl, C₁₋₆alkoxy and methylenedioxy, and heteroaryl means thienyl, flryl or pyridyl each optionally substituted by one or more (e.g. 1, 2 or 3) substituents selected from halogen, C₁₋₆ alkyl and C₁₋₆alkoxy;
R² represents a monocyclic aromatic ring selected from benzene, optionally substituted by one or more (e.g., 1, 2 or 3) atoms or groups comprising halogen, hydroxy, C₁₋₆alkyl, C₁₋₆alkoxy, —CO₂R^{b}, haloC₁₋₆alkyl, haloC₁₋₆alkoxy, cyano, nitro and NR^{a}R^{b}, or R² represents an optionally substituted thiophene, fuiran, pyridine, or a bicyclic ring 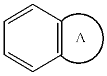
attached to the rest of the molecule via one of the benzene ring carbon atoms and wherein the fiused ring A is a 5- or 6-membered ring which may be saturated or partially or filly unsaturated and comprises carbon atoms and optionally one or two heteroatoms selected from oxygen, sulphur and nitrogen; where optional substitution means one or more (e.g. 1, 2 or 3) atoms or groups comprising halogen, C₁₋₆alkyl, C₁₋₆alkoxy and arylC₁₋₃aLkyl as defined above;
R³ represents hydrogen or C₁₋₃ alkyl, or R¹ and R³ together represent a 3- or 4-membered alkyl or alkenyl chain; and
R^{a} and R_{b} are each hydrogen or C₁₋₆alkyl, or Rₐ may also represent C₂₋₇alkanoyl or C₁₋₆alkylsulphonyl.

20. The method according to claim 19 wherein said compounds are selected from the group consisting of cis-2,3,6,7,12,12a-hexahydro-2-(4-pyridylmethyl)-6-(3 ,4-methylenedioxyphenyl)pyrazino(2′,1′:6,1)pyrido(3 ,4-b)indole-1,4-dione; cis-2,3,6,7,12,12a-hexahydro-6-(2,3-dihydrobenzo:b)furan-5-yl)-2-methylpyrazino(2′,1′:6,1)pyrido(3,4-b)indole-1,4-dione; cis-2,3,6,7,12,12a-hexahydro-6-(5-bromo-2-thienyl)-2-methylpyrazino(2′,1′:6,1)pyrido(3,4-b)indole-1,4-dione; cis-2,3,6,7,12,12a-hexahydro-2-butyl-6-(4-methylphenyl)pyrazino(2′,1′:6,1)pyrido(3,4-b)indole-1,4-dione; (6R, 12aR)-2,3,6,7,12,12a-hexahydro-2-isopropyl-6-(3,4-methylenedioxyphenyl)pyrazino(2′,1′:6,1)pyrido(3,4-b)indole-1,4-dione; (6R,12aR)-2,3,6,7,12,12a-Hexahydro-2-cyclopentyl-6-(3,4-methylenedioxyphenyl)-pyrazino(2′,1′:6,1)pyrido(3,4-b)indole-1,4-dione; (6R,12aR)-2,3,6,7,12,12a-hexahydro-2-cyclopropylethyl-6-(4-methoxyphenyl)pyrazino(2′,1′:6,1)pyrido(3,4-b)indole-1,4-dione; (6R, 12aR)-2,3,6,7,12,12a-hexahydro-6-(3-chloro-4-methoxyphenyl)-2-methy lpyrazino(2′,1′:6,1)pyrido(3,4-b)indole-1,4-dione; (6R, 12aR)-2,3,6,7,12,12a-hexahydro-2-methyl-6-(3,4-methylenedioxyphenyl)pyrazino(2′,1′:6,1)pyrido(3,4-b)indole-1,4-dione; (6R, 12aR)-2,3,6,7,12,12a-Hexahydro-6-(3,4-methylenedioxyphenyl)-pyrazino(2′,1′:6,1)pyrido (3,4-b) indole-1,4-dione; (5aR, 12R, 14aS)-1,2,3,5,6,1,12,14a-octahydro-12-(3,4-methylenedioxyphenyl)-pyrrolo(1″,2″:4′,5′)pyrazino(2′,1′:6,1)pyrido(3,4-b)indole-5-1,4-dione; and physiologically acceptable salts and solvates thereof.

21. The method according to claim 20 wherein the compound is selected from (6R,12aR)-2,3,6,7,12,12a-hexahydro-2-methyl-6-(3,4-methylenedioxyphenyl)-pyrazino(2′,1′:6,1)pyrido(3,4-b)indole-1,4-dione; and physiologically acceptable salts and solvates thereof.

22. A method for inhibiting the growth of neoplastic cells comprising exposing the cells to a growth inhibiting effective amount of a compound of Formula I or pharmaceutically acceptable salt thereof 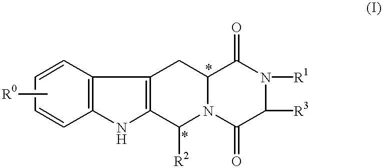
wherein R⁰ represents hydrogen, halogen or C1-6 alkyl;
R¹ represents hydrogen, C₁₋₆ alkyl, C₂₋₆alkenyl, C₂₋₆ alkynyl, haloC₁₋₆ alkyl, C₃₋₈cycloalkyl, C₃₋₈cycloalkylC₁₋₃alkyl, arylC₁₋₃alkyl or heteroarylC₁₋₃alkyl, where aryl means phenyl or phenyl substituted by one or more (e.g. 1, 2 or 3) substituents selected from halogen, C₁₋₆alkyl, C₁₋₆alkoxy and methylenedioxy, and heteroaryl means thienyl, flryl or pyridyl each optionally substituted by one or more (e.g. 1, 2 or 3) substituents selected from halogen, C1-6 alkyl and C₁₋₆alkoxy;
R² represents a monocyclic aromatic ring selected from benzene, optionally substituted by one or more (e.g., 1, 2 or 3) atoms or groups comprising halogen, hydroxy, C₁₋₆alkyl, C₁₋₆alkoxy, —CO₂R^{b}, haloC₁₋₆alkyl, haloC₁₋₆alkoxy, cyano, nitro and NR^{a}R^{b}, or R² represents an optionally substituted thiophene, furan, pyridine, or a bicyclic ring 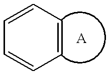
attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen; where optional substitution means one or more (e.g. 1, 2 or 3) atoms or groups comprising halogen, C₁₋₆alkyl, C₁₋₆alkoxy and arylC₁₋₃alkyl as defined above;
R³ represents hydrogen or C₁₋₃ alkyl, or R¹ and R³ together represent a 3- or 4-membered alkyl or alkenyl chain; and
R^{a} and R^{b} are each hydrogen or C₁₋₆alkyl, or Rₐ may also represent C₂₋₇alkanoyl or C₁₋₆alkylsulphonyl.

23. The method accordimg to claim 22 wherein said compounds are selected from the group consisting of cis-2,3,6,7,12,12a-hexahydro-2-(4-pyridylmethyl)-6-(3,4-methylenedioxyphenyl)pyrazino(2′,1′:6,1)pyrido(3,4-b)indole-1,4-dione; cis-2,3,6,7,12,12a-hexahydro-6-(2,3-dihydrobenzocb)furan-5-yl)-2-methylpyralzino(2′,1′:6,1)pyrido(3,4-b)indole-1,4-dione; cis-2,3,6,7,12,12a-hexahydro-6-(5-bromo-2-thienyl)-2-methylpyrazino(2′,1′:6,1)pyrido(3,4-b)indole-1,4-dione; cis-2,3,6,7,12,12a-hexahydro-2-butyl-6-(4-methylphenyl)pyrazino(2′,1′:6,1)pyrido(3,4-b)indole-1,4-dione; (6R,12aR)-2,3,6,7,12,12a-hexahydro-2-isopropyl-6-(3,4-methylenedioxyphenyl)pyrazino(2′,1′:6,1)pyrido(3,4-b)indole-1,4-dione; (6R,12aR)-2,3,6,7,12,12a-Hexahydro-2-cyclopentyl-6-(3,4-methylenedioxyphenyl)-pyrazino(2′,1′:6,1)pyrido(3,4-b)indole-1,4-dione; (6R,12aR)-2,3,6,7,12,12a-hexahydro-2-cyclopropylmethyl-6-(4-methoxyphenyl)pyrazino(2′,1′:6,1)pyrido(3,4-b)indole-1,4-dione; (6R,12aR)-2,3,6,7,12,12a-hexahydro-6-(3-chloro-4-methoxyphenyl)-2-methy lpyrazino(2′,1′:6,1)pyrido(3,4-b)indole- 1,4-dione; (6R,12aR)-2,3,6,7,12,12a-hexahydro-2-methyl-6-(3,4-methylenedioxyphenyl)pyrazino(2′,1′:6,1)pyrido(3,4-b)indole-1,4-dione; (6R,12aR)-2,3,6,7,12,12a-Hexahydro-6-(3,4-methylenedioxyphenyl)-pyrazino(2′,1′:6,1)pyrido (3,4-b) indole-1,4-dione; (5aR, 12R, 14aS)-1,2,3,5,6,1,12,14a-octahydro-12-(3,4-methylenedioxyphenyl)-pyrrolo(1″,2″:4′,5′)pyrazino(2′,1′:6,1)pyrido(3,4-b)indole-5-1,4-dione; and physiologically acceptable salts and solvates thereof.

24. The method according to claim 23 wherein the compound is selected from (6R, 12aR)-2,3,6,7,12,12a-hexahydro-2-methyl-6-(3,4-methylenedioxyphenyl)-pyrazino(2′,1′:6,1)pyrido(3,4-b)indole-1,4-dione; and physiologically acceptable salts and solvates thereof.

25. A method for regulating apoptosis in human cells comprising exposing said cells to an effective amount of a compouind of formula: 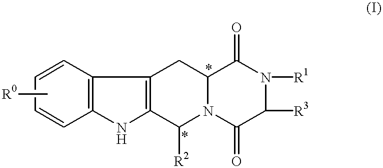
wherein R⁰ represents hydrogen, halogen or C₁₋₆ alkyl;
R¹ represents hydrogen, C₁₋₆ alkyl, C₂₋₆alkenyl, C₂₋₆ alkynyl, haloC₁₋₆ alkyl, C₃₋₈cycloalkyl, C₃₋₈cycloalkylC₁₋₃alkyl, arylC₁₋₃alkyl or heteroarylC₁₋₃alkyl, where aryl means phenyl or phenyl substituted by one or more (e.g. 1, 2 or 3) substituents selected from halogen, C₁₋₆alkyl, C₁₋₆alkoxy and methylenedioxy, and heteroaryl means thienyl, furyl or pyridyl each optionally substituted by one or more (e.g. 1, 2 or 3) substituents selected from halogen, C₁₋₆ alkyl and C₁₋₆alkoxy;
R² represents a monocyclic aromatic ring selected from benzene, optionally substituted by one or more (e.g., 1, 2 or 3) atoms or groups comprising halogen, hydroxy, C₁₋₆alkyl, C₁₋₆alkoxy, —CO₂R^{b}, haloC₁₋₆alkyl, haloC₁₋₆alkoxy, cyano, nitro and NR^{a}R^{b}, or R² represents an optionally substituted thiophene, furan, pyridine, or a bicyclic ring 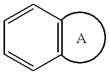
attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen; where optional substitution means one or more (e.g. 1, 2 or 3) atoms or groups comprising halogen, C₁₋₆alkyl, C₁₋₆alkoxy and arylC₁₋₃alkyl as defined above;
R³ represents hydrogen or C₁₋₃ alkyl, or R¹ and R³ together represent a 3- or 4-membered alkyl or alkenyl chain; and
R^{a} and R^{b} are each hydrogen or C₁₋₆alkyl, or Rₐ may also represent C₂₋₇alkanoyl or C₁₋₆alkylsulphonyl.

26. The method according to claim 25 wherein said compounds are selected from the group consisting of cis-2,3,6,7,12,12a-hexahydro-2-(4-pyridylmethyl)-6-(3,4-methylenedioxyphenyl)pyrazino(2′,1′:6,1)pyrido(3,4-b)indole-1,4-dione; cis-2,3,6,7,12,12a-hexahydro-6-(2,3-dihydrobenzofb)furan-5-yl)-2-methylpyrazino(2′,1′:6,1)pyrido(3,4-b)indole-1,4-dione; cis-2,3,6,7,12,12a-hexahydro-6-(5-bromo-2-thienyl)-2-methylpyrazino(2′,1′:6,1)pyrido(3,4-b)indole-1,4-dione; cis-2,3,6,7,12,12a-hexahydro-2-butyl-6-(4-methylphenyl)pyrazino(2′,1′:6,1)pyrido(3,4-b)indole-1,4-dione; (6R,12aR)-2,3,6,7,12,12a-hexahydro-2-isopropyl-6-(3,4-methylenedioxyphenyl)pyrazino(2′,1′:6,1)pyrido(3,4-b)indole-1,4-dione; (6R,12aR)-2,3,6,7,12,12a-Hexahydro-2-cyclopentyl-6-(3,4-methylenedioxyphenyl)-pyrazino(2′,1′:6,1)pyrido(3,4-b)indole-1,4-dione; (6R,12aR)-2,3,6,7,12,12a-hexahydro-2-cyclopropylnethyl-6-(4-methoxyphenyl)pyrazino(2′,1′:6,1)pyrido(3,4-b)indole-1,4-dione; (6R,12aR)-2,3,6,7,12,12a-hexahydro-6-(3-chloro-4-methoxyphenyl)-2-methy lpyrazino(2′,1′:6,1)pyrido(3,4-b)indole-1,4-dione; (6R,12aR)-2,3,6,7,12,12a-hexahydro-2-methyl-6-(3,4-methylenedioxyphenyl)pyrazino(2′,1′:6,1)pyrido(3,4-b)indole-1,4-dione; ( 6R,12aR)-2,3,6,7,12,12a-Hexahydro-6-(3,4-methylenedioxyphenyl)-pyrazino(2′,1′:6,1)pyrido (3,4-b) indole-1,4-dione; (5aR, 12R, 14aS)-1,2,3,5,6,1,12,14a-octahydro-12-(3,4-methylenedioxyphenyl)-pyrrolo(1″,2″:4′,5′)pyrazino(2′,1′:6,1)pyrido(3,4-b)indole-5-1,4-dione; and physiologically acceptable salts and solvates thereof.

27. The method according to claim 26 wherein the compound is selected from (6R,12aR)-2,3,6,7,12,12a-hexahydro-2-methyl-6-(3,4-methylenedioxyphenyl)-pyrazino(2′,1′:6,1)pyrido(3,4-b)indole-1,4-dione; and physiologically acceptable salts and solvates thereof.,
